(19) United States
(12) Patent Application Publication
(10) Pub. No.: US 2019/0360980 A1
(43) Pub. Date: Nov. 28, 2019

(54) METHODS OF ANALYZING PLURALITIES OF CELLS AND DETECTING PROTEIN SEQUENCE VARIANTS IN BIOLOGICAL PRODUCT MANUFACTURING

(71) Applicant: LONZA LTD, Visp (CH)

(72) Inventor: James GRAHAM, Oxford (GB)

(73) Assignees: LONZA LTD, Visp (CH); LONZA LTD, Visp (CH)

(21) Appl. No.: 16/483,141

(22) PCT Filed: Feb. 2, 2018
(86) PCT No.: PCT/US2018/016555

§ 371 Date: Aug. 2, 2019

Related U.S. Application Data
(60) Provisional application No. 62/454,567, filed on Feb. 3, 2017.
(60) Provisional application No. 62/464,775, filed on Feb. 28, 2017.
(60) Provisional application No. 62/510,559, filed on May 24, 2017.

(51) Int. Cl. G01N 30/72; G01N 33/50; C12Q 1/6872; G01N 33/68; G16B 40/10

(52) U.S. Cl. CPC G01N 30/72; G01N 33/5005; G01N 2333/70596; G01N 33/6848; G16B 40/10; C12Q 1/6872

(57) ABSTRACT

Disclosed are methods for detecting protein sequence variants and evaluating the probability of generating protein sequence variants in biological product manufacturing.

### RELATED APPLICATIONS

This application claims priority to U.S. Ser. No. 62/454,567, filed Feb. 3, 2017, U.S. Ser. No. 62/464,775, filed Feb. 28, 2017, and U.S. Ser. No. 62/510,559, filed May 24, 2017, the contents of each of which are incorporated herein by reference in their entireties.

### FIELD OF THE INVENTION

The present disclosure relates to methods and systems for evaluating the incidence of protein sequence variants at various stages of biological product manufacturing.

### BACKGROUND

A protein sequence variant (PSV) is defined as unintended amino acid sequence change which can occur as a result of a genomic nucleotide change or a translational misincorporation. Low level sequence variants contribute to product heterogeneity which may affect product efficacy and immunogenicity. Incorporation of a methodology for systematic screening for sequence variants into the stable cell line development process is important for successful manufacturing of biopharmaceuticals.

Several potential mechanisms have been reported for how amino acid sequence variants can arise, including mutation of genomic DNA, mistranslation at specific codons and nutrient depletion. Systematic screening for sequence variants is emerging as an integral analytical component of cell line construction process for successful manufacturing of biopharmaceuticals.

One approach is to use Amplicon sequencing, a sensitive method used to identify mutation in the nucleic acids. A correct sequence of DNA or RNA does not however guarantee a correct protein sequence and the error rate for translation process is known to be much higher. Error rates in translation (10⁻⁴-10⁻³) are generally thought to be about an order of magnitude higher than those in transcription (10⁻⁵-10⁻⁴). For the above reasons it can be important that the analysis of sequence variants is performed at the protein level. Peptide mapping analysis with LC-MS offers excellent specificity and sensitivity for in-depth characterisation of a protein sequence. Sequence variants can be detected by de novo analysis of MS2 data. The sensitivity of the method relies on very high quality of fragmentation data being generated for low abundance species. A disadvantage of this methodology is high level of false positive.

Thus a need exists for improved systematic evaluation of protein product variants at various stages of biopharmaceutical manufacturing.

### SUMMARY OF THE INVENTION

In one aspect, the invention features a method of analysing a plurality of cells, a method using the plurality of cells, or a polypeptide made by the plurality of cells, comprising:

a) culturing a plurality of cells, at least one cell of the plurality of cells comprising a nucleic acid sequence encoding a product comprising a first amino acid sequence, e.g., a production sequence, to make conditioned media comprising product;

b) subjecting a first sample of polypeptide from the conditioned media comprising product to a first sequence-based reaction, e.g., digestion with a proteolytic enzyme, to provide a first reaction product, e.g., a proteolytic fragment (and, optionally, e.g., subjecting the reaction product to a separation step, e.g., by mass spec);

c) comparing a value for the first reaction product, e.g., presence, mobility (e.g., time of flight) or molecular weight, with a reference value, e.g., a value for a reaction product produced by application of the first sequence-based reaction to a reference sequence, e.g., the first amino acid sequence, and responsive to the comparison, selecting a reaction product component for further analysis, e.g., sequencing;

d) subjecting a second sample of polypeptide from the conditioned media comprising product to a second sequence-based reaction, e.g., digestion with a second proteolytic enzyme, to provide a second reaction product, e.g., a proteolytic fragment (and, optionally, e.g., subjecting the reaction product to a separation step, e.g., by mass spec);

e) comparing a value for the second reaction product, e.g., presence, mobility (e.g., time of flight) or molecular weight, with a reference value, e.g., a value for a reaction product produced by application of the second sequence-based reaction to a reference sequence, e.g., the first amino acid sequence, and responsive to the comparison, selecting a reaction product component for further analysis, e.g., sequencing;

f) optionally, subjecting a third sample of polypeptide from the conditioned media comprising product to a third sequence-based reaction, e.g., digestion with a proteolytic enzyme, to provide a third reaction product, e.g., a proteolytic fragment (and, optionally, e.g., subjecting the reaction product to a separation step, e.g., by mass spec);

g) optionally, comparing a value for the third reaction product, e.g., presence, mobility (e.g., time of flight) or molecular weight, with a reference value, e.g., a value for a reaction product produced by application of the third sequence-based reaction to a reference sequence, e.g., the first sequence, and responsive to the comparison, selecting a reaction product component for further analysis, e.g., sequencing,

h) optionally, responsive to the results of c) and optionally e) and/or g), determining if a sequence other than the first amino acid sequence is present in the plurality of cells,

thereby analysing a plurality of cells, a method using the plurality of cells, or a polypeptide made by the plurality of cells.

In another aspect, the invention features a method of detecting a protein sequence variant, the method comprising:

a) providing a population of cells, wherein the cells produce a protein product;

b) purifying the protein product from the population of cells;

c) preparing the purified protein product for analysis by mass spectrometry;

d) analyzing the prepared purified protein product by mass spectrometry;

wherein a)-d) are repeated, in parallel or consequentially, for a plurality (e.g., more than one, e.g., two, three, four, five, six, seven, eight, nine, ten or more) of populations of cells; and

e) detecting protein sequence variants by comparing mass spectrometry data from the plurality of populations of cells and a database of mass spectrometry data,

thereby detecting the protein sequence variant.

In another aspect, the invention features a method of analysing a plurality of cells, the method comprising:

a) culturing a plurality of cells, at least one cell of the plurality of cells comprising a nucleic acid sequence encoding a product, said product comprising a first amino acid sequence, to make conditioned media comprising product;

b) subjecting a first sample of polypeptide from the conditioned media comprising product to a first sequence-based reaction to provide a first reaction product;

c) comparing a value for the first reaction product with a reference value, and responsive to the comparison, selecting a reaction product component for further analysis;

d) subjecting a second sample of polypeptide from the conditioned media comprising product to a second sequence-based reaction to provide a second reaction product;

e) comparing a value for the second reaction product with a reference value, and responsive to the comparison, selecting a reaction product component for further analysis;

f) optionally, subjecting a third sample of polypeptide from the conditioned media comprising product to a third sequence-based reaction to provide a third reaction product;

g) optionally, comparing a value for the third reaction product with a reference value, and responsive to the comparison, selecting a reaction product component for further analysis,

h) responsive to the results of c) and optionally e) and g), determining if a sequence other than the first amino acid sequence is present in the plurality of cells,

thereby analysing a plurality of cells.

In another aspect, the invention features a method of detecting a protein sequence variant, the method comprising:

a) providing purified protein product from culture media comprising a population of cells, e.g., a plurality of cells, wherein the cells produce a protein product;

b) analyzing the purified protein product by mass spectrometry;

wherein a)-b) are repeated, in parallel or sequentially, for a plurality of samples within the same population of cells or different populations of cells; and

c) detecting protein sequence variants within the plurality of samples by comparing mass spectrometry data from the plurality of samples and a database of mass spectrometry data,

thereby detecting the protein sequence variant.

In some embodiments, the sample is an aliquot.

In another aspect, the invention features a polypeptide made, e.g., by any of the methods described herein, or by the plurality of cells or population of cells of any of the methods described herein.

### BRIEF DESCRIPTION OF THE FIGURES

FIG. 1 is a workflow of protein sequence variant analysis.

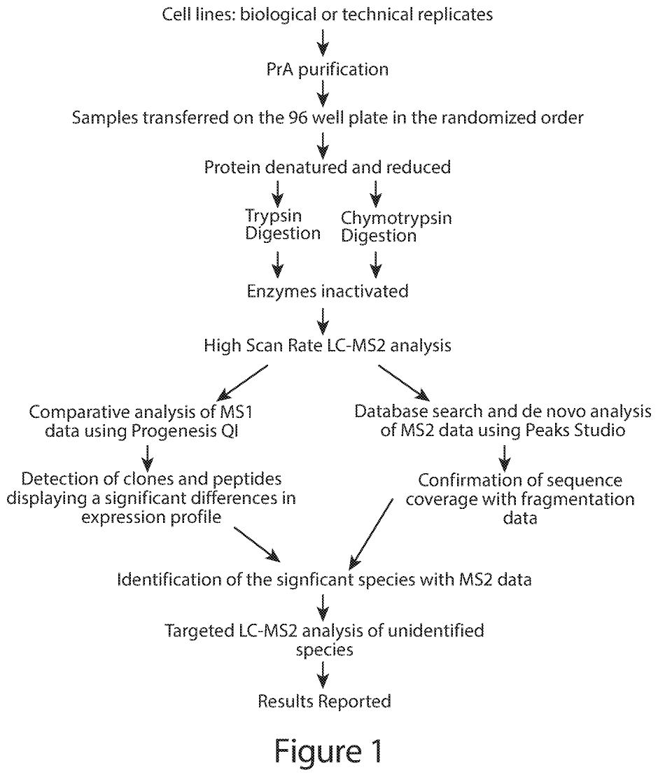

FIG. 2 shows the effect of urea molarity on trypsin efficiency for digestion of rituximab.

FIG. 3A shows the effect of urea molarity and temperature on trypsin efficiency in terms of the number of missed cleaved peptides of trastuzumab. FIG. 3B shows the same data in table form.

FIG. 4 shows the effect of urea molarity and temperature on digestion efficiency of GFYPSDIAVEWESNGQPENNYK peptide.

FIG. 5 shows the effect of urea molarity on activity of chymotrypsin for digestion of rituximab.

FIG. 6A shows the effect of urea molarity and temperature on incomplete digestion of trastuzumab using chymotrypsin. FIG. 6B is a table showing the data of 6A.

FIG. 7 shows the effect of urea molarity and temperature on the efficiency of chymotrypsin digestion of trastuzumab in 2M urea at 37° C. and in 0.5M urea at 25° C.

FIG. 8 shows the effect of urea molarity on AspN efficiency for digestion of rituximab.

FIG. 9 shows the effect of urea molarity on AspN efficiency for digestion of cB72.3.

FIG. 10 is a coverage plot for combined tryptic/chymotryptic digestion of trastuzumab HC region with nanoLC-MS2 analysis with Orbitrap Fusion. One tripeptide and one single residue peptide were not detected in the heavy chain (red circles).

FIG. 11 is a coverage plot for combined tryptic/chymotryptic/lysC digestion of trastuzumab HC region with nanoLC-MS2 analysis with Orbitrap Fusion.

FIG. 12 shows a workflow of protein sequence variant analysis of model protein rituximab.

FIG. 13 shows an abundance profile for potential sequence variant detected in late generation clone 4B04.

FIG. 14 shows an MS profile for a potential sequence variant.

FIG. 15 shows a targeted MS/MS analysis of a potential sequence variant.

FIG. 16 shows an example LC system compatible with a wash procedure described herein.

FIG. 17 shows example protocols for the analytical gradient and cleaning gradient for use in a wash protocol. Arrows indicate which colors correspond to which pumps.

FIG. 18 shows a diagram of a plate for use in a buffer stability screen.

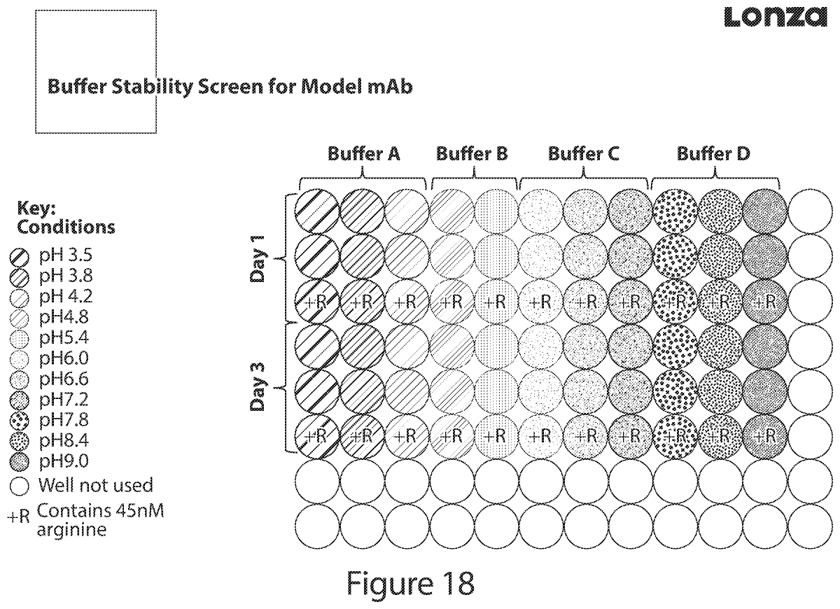

FIG. 19 shows a graph of aggregation across buffer pH for Day 1 (without arginine), Day 1 (with arginine), Day 3 (without arginine), and Day 3 (with arginine).

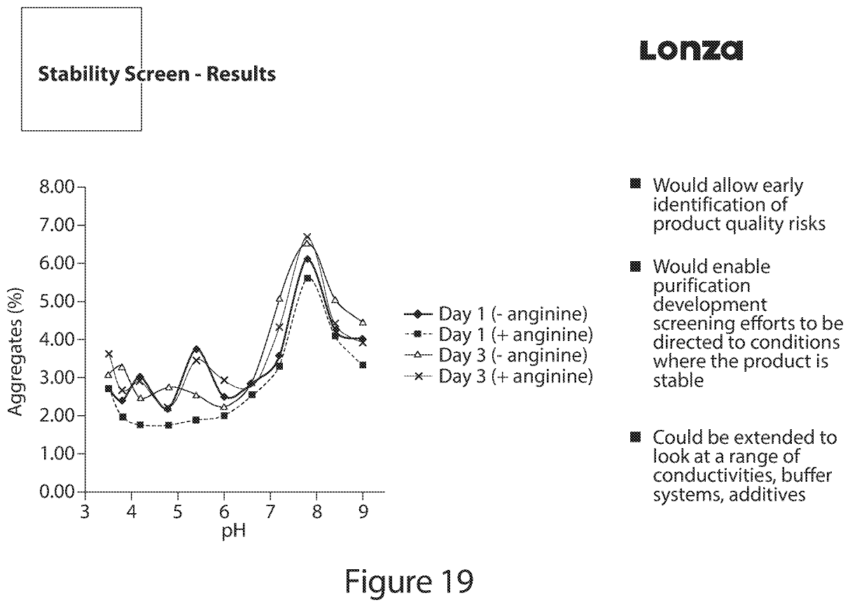

FIG. 20 shows a workflow of protein sequence variant analysis.

FIG. 21 shows MS profiles for identified sequence variants.

FIG. 22 shows a targeted MS/MS analysis of a sequence variant at top and an abundance profile for the sequence variant at bottom.

FIG. 23 shows a 3d spectrum of the MS/MS data for S160C variant at top and the trastuzumab variant sequence and a trypsin cleavage fragment sequence of the same at bottom.

FIG. 24 shows MS/MS analysis of spiked sequence variants.

### DETAILED DESCRIPTION OF THE INVENTION

### Definitions

The articles “a” and “an” are used herein to refer to one or to more than one (i.e., to at least one) of the grammatical object of the article. By way of example, “a cell” can mean one cell or more than one cell.

As used herein, the term “aliquot” refers to a volume of a solution, e.g., of purified protein, prepared purified protein, culture medium, or a conditioned culture medium. In an embodiment, each aliquot satisfies a condition with regard to volume, e.g., each aliquot has: a minimal volume, e.g., a preset minimal value; falls within a range between a minimal and a maximal value, e.g., a preset minimal and/or maximal value; approximately equal values, e.g., a preset value; or the same volume, e.g., a preset value. When a larger amount of a liquid, e.g., a conditioned culture medium, is divided into a plurality of aliquots, the plurality may be equal to the entire larger amount, or to less than the entire larger amount.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

As used herein, the term “plurality of aliquots” refers to more than one (e.g., two or more) aliquots.

As used herein, the term “endogenous” refers to any material from or naturally produced inside an organism, cell, tissue or system.

As used herein, the term “exogenous” refers to any material introduced to or produced outside of an organism, cell, tissue or system. Accordingly, “exogenous nucleic acid” refers to a nucleic acid that is introduced to or produced outside of an organism, cell, tissue or system. In an embodiment, sequences of the exogenous nucleic acid are not naturally produced, or cannot be naturally found, inside the organism, cell, tissue, or system that the exogenous nucleic acid is introduced into. Similarly, “exogenous polypeptide” refers to a polypeptide that is not naturally produced, or cannot be naturally found, inside the organism, cell, tissue, or system that the exogenous polypeptide is introduced to, e.g., by expression from an exogenous nucleic acid sequence.

As used herein, the term “heterologous” refers to any material from one species, when introduced to an organism, cell, tissue or system from a different species.

As used herein, the terms “nucleic acid,” “polynucleotide,” or “nucleic acid molecule” are used interchangeably and refers to deoxyribonucleic acid (DNA) or ribonucleic acid (RNA), or a combination of a DNA or RNA thereof, and polymers thereof in either single- or double-stranded form. The term “nucleic acid” includes, but is not limited to, a gene, cDNA, or an mRNA. In one embodiment, the nucleic acid molecule is synthetic (e.g., chemically synthesized or artificial) or recombinant. Unless specifically limited, the term encompasses molecules containing analogues or derivatives of natural nucleotides that have similar binding properties as the reference nucleic acid and are metabolized in a manner similar to naturally or non-naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others.

“Product” as that term is used herein refers to a molecule, e.g., polypeptide, e.g., protein, e.g., glycoprotein, nucleic acid, lipid, saccharide, polysaccharide, or any hybrid thereof, that is produced, e.g., expressed, by a cell, e.g., a cell which has been modified or engineered to produce the product. In an embodiment, the product is a protein or polypeptide product. In one embodiment, the product comprises a naturally occurring product. In an embodiment the product comprises a non-naturally occurring product. In one embodiment, a portion of the product is naturally occurring, while another portion of the product is non-naturally occurring. In one embodiment, the product is a polypeptide, e.g., a recombinant polypeptide. In one embodiment, the product is suitable for diagnostic or pre-clinical use. In another embodiment, the product is suitable for therapeutic use, e.g., for treatment of a disease. In some embodiments, a product is a protein product. In some embodiments, a product is a recombinant or therapeutic protein described herein, e.g., in Tables 1-4.

As used herein, “sequence variant,” “protein sequence variant,” “protein product sequence variant,” or similar term refers to a species of protein product which differs from a reference protein product. E.g., a protein comprising an amino acid sequence different from a reference amino acid sequence. Typically, the sequence variant occurs as a result of a genomic nucleotide change or translational misincorporation. For example, a sequence variant of a protein may comprise zero, one, or more of each of the following amino acid sequence alterations: a substitution, a deletion, and an insertion.

As used herein, the terms “plurality of sequence variants”, “plurality of protein sequence variants” and similar refer to more than one (e.g., two or more) sequence variants, protein sequence variants, etc.

As used herein, a plurality of cells or a population of cells (used interchangeably) refer to more than one (e.g., two or more) cells. In an embodiment, a plurality of cells may comprise cells of a cell line, e.g., clonal cells. In an embodiment, a plurality of cells may comprise cells of a mixture of cell lines, e.g., cells from different clonal lineages. In an embodiment, a plurality of cells may primarily comprise (e.g., the plurality comprises greater than 50, 60, 70, 80, 90, 95, or 99%) cells of a cell line, e.g., clonal cells. In some embodiments, at least one cell of a plurality of cells comprises a first sequence, e.g., a production sequence, e.g., a sequence encoding a recombinant protein product. In some embodiments, the majority of cells in a plurality of cells comprise a first sequence, e.g., a production sequence, e.g., a sequence encoding a recombinant protein product. In some embodiments, each cell in a plurality of cells comprises a first sequence, e.g., a production sequence, e.g., a sequence encoding a recombinant protein product. In some embodiments, at least one cell of a plurality of cells is capable of producing a polypeptide encoded by a first sequence, e.g., the polypeptide encoded by a production sequence, e.g., a recombinant protein product. In some embodiments, a plurality of populations of cells refers to more than one (e.g., two or more) populations of cells.

As used herein, a sequence-based reaction is a reaction performed on a polypeptide that processes the polypeptide based on the polypeptide's amino acid sequence, producing one or more (e.g., one, two, three, four, five, six . . . , one hundred, or more) reaction products. In some embodiments, the sequence-based reaction is digestion by a protease or proteolytic enzyme. In some embodiments, the protease or proteolytic enzyme recognizes a specific sequence of amino acids and cleaves a site within, adjacent to, or at a distance to the specific sequence of amino acids. As used herein, a reaction product is the product of a sequence-based reaction. In some embodiments, a reaction product is one or more portions of a polypeptide, e.g., one or more fragments, e.g., one or more proteolytic fragments. In some embodiments, a reaction product is of a molecular weight suitable for further analysis, e.g., analysis by mass spectrometry, e.g., LC/MS or MS/MS. In some embodiments, a component of a reaction product or a reaction product component is a single portion of a polypeptide produced by a sequence-based reaction, e.g., a single fragment, e.g., a single proteolytic fragment.

As used herein, a value for the reaction product refers to a value of a parameter related to the reaction product. In some embodiments, parameters related to the reaction product include presence, mobility (e.g., time of flight, e.g., time of flight in a mass spectrometer; or migration rate in a chromatographic technique), molecular weight, charge, ionizability, or the presence of a label.

### Detection of Protein Sequence Variants

In one aspect, the invention of the disclosure relates to a method for detecting protein sequence variants in plurality of cells, e.g., cell lines designed to produce protein products.

The current procedure for characterisation of protein's primary structure in Lonza (tryptic peptide mapping by LC-MSMS, UKSL-8092) is designed to confirm the theoretical product sequence. The detectability of unintended protein variants is limited by the resolving capacity of the chromatographic method. The scope of the protein sequence variant analysis (PSVA) is detection and identification of multiple amino acid substitutions, N- and C-terminal extension and truncation.

Sequence variants were detected in comparative screening of peptide map MS1 data by application of multivariate analysis and MS2 data were used for identification of the significantly different species.

In some embodiments, sequence variants detected by the methods described herein are further analysed using in silico immunogenicity evaluation tools. The immunogenicity of a possible protein sequence variant may have effects on downstream therapeutic efficacy and product reliability. In silico tools can be used to evaluate the binding of sequence variants or fragments thereof to elements of the immune system, as well as their propensity to provoke an immune response. Methods compatible with the in silico evaluation of immunogenicity of protein sequence variants and with the methods of the present invention can be found in U.S. Pat. No. 7,702,465 and European patent 1516275, hereby incorporated by reference in their entirety, as well as commercially (e.g., Epibase by Lonza).

In some embodiments, sequence variants detected by the methods described herein are further analysed to predict protein aggregation, e.g., propensity/likelihood of protein aggregation. Protein aggregation is a commonly encountered problem during biopharmaceutical development. It can potentially occur at several different steps of the manufacturing process such as fermentation, purification, formulation and storage. The impact of aggregation spans not only the manufacturing process but also the target product profile, delivery and critically, patient safety (Vazquez-Rey and Lang. (2011) Biotechnol. Bioeng. 108. 1494-508). An aggregating product can increase manufacturing costs, lengthen development timelines and limit the options for formulation and delivery. Aggregation depends both on the intrinsic properties of the protein (intrinsic aggregation propensity) and on environmental factors such as pH, concentration, buffers, excipients and shear-forces. However, the fundamental difference as to why one antibody aggregates during a process step or manufacturing and others do not is encoded in their amino-acid sequence and intrinsic aggregation propensities. Prediction method: Sentinel APART™ was developed using machine learning algorithms based on sequence and structural features of antibodies as descriptors (Obrezanova et al. (2015). MAbs. 7. 352-363). The model was trained and tested on a set of sequence-diverse antibodies, designed to cover a wide physico-chemical descriptor space and to contain low and high expressing as well as aggregating and non-aggregating antibodies. The characteristics of all antibodies in the set were experimentally determined at Lonza.

In some embodiments, sequence variants detected by the methods described herein are further analysed to detect deamidation. (Asparagine) deamidation is a non-enzymatic reaction which over time produces a heterogeneous mixture of molecules with Asparagine, isoApartate or Aspartate (Aspartic acid) at the affected position. Deamidation is caused by hydrolysis of the amide group on the side-chains of Asparagine and Glutamine. Three primary factors influence the deamidation rates of peptides: pH, high temperature and primary sequence. The secondary and tertiary structures of protein can also significantly alter the deamidation rate. (Both Asparagine and Glutamine are susceptible to deamidation. In reality we only concern ourselves with a subset of Asparagine sites, where the next residue is small and hydrophilic. It is possible to rewrite this section so that it applies both to Asparagine and Glutamine) In addition to causing charge heterogeneity, (Asparagine) deamidation can affect protein function if it occurs in a binding interface such as in antibody CDRs (Harris et al. (2001). J. Chromatogr. B. Biomed. Sci Appl. 752. 233-245). Deamidation can lead to subsequent issues related to fragmentation, aggregation and immunogenicity (Vlasak and Ionescu. (2011). MAbs. 3. 253-263; Doyle et al. (2007). Autoimmunity. 40. 131-137; Dunkelberger. (2012). J. Am. Chem. Soc. 134. 12658-12667). Asparagine residues that are prone to deamidation as determined by a combination of primary (Robinson and Robinson, 2001; Liu, Hui F., et al. “Recovery and purification process development for monoclonal antibody production.” MAbs. Vol. 2. No. 5. Taylor & Francis, 2010) and tertiary structure analysis are predicted to be liabilities.

In some embodiments, sequence variants detected by the methods described herein are further analysed to detect aspartic acid isomerisation and fragmentation. Aspartic acid isomerisation is the non-enzymatic interconversion of Aspartic acid and isoAspartic acid residues. The peptide bond C-terminal to Aspartic acid can be susceptible to fragmentation in acidic conditions. These reactions proceed through intermediates similar to those of the Asparagine deamidation reaction. The rate of Aspartic acid isomerisation and fragmentation is influenced by pH, temperature and primary sequence. The secondary and tertiary structures of protein can also alter the rate. Aspartic acid isomerisation can affect protein function when it occurs in binding interfaces such as in antibody CDRs (Harris et al. (2001)). Isomerisation also causes charge heterogeneity and can result in fragmentation caused by cleavage of the peptide back-bone. The fragmentation reaction primarily occurs at a low pH and Asp-Pro peptide bonds are more labile than other peptide bonds (Vlasak and Ionescu. (2011)). Aspartic acid isomerisation has the potential to increase immunogenicity (Doyle et al. (2007)), a risk that is further increased as fragmentation favours aggregate formation. Aspartic acid residues at risk of isomerisation and/or fragmentation are detected using a combination of primary and tertiary structure analysis.

In some embodiments, sequence variants detected by the methods described herein are further analysed to detect C-terminal lysine processing. C-terminal Lysine processing is a common modification in antibodies and other proteins that occurs during bioprocessing likely due to the action of basic carboxypeptidases (Cai et al. (2011). Biotechnol. Bioeng. 108. 404-412). C-terminal Lysine processing is a major source of charge and mass heterogeneity in antibody products as species with two, one or no Lysines can be formed. C-terminal Lysine processing is a source of mass and charge heterogeneity but is not known to affect antibody potency or the safety profile. C-terminal Lysines are detected.

In some embodiments, sequence variants detected by the methods described herein are further analysed to predict Fc ADCC/CDC response, half-life, and protein A purification. The antibody fragment crystallisable (Fc) contains the regions responsible for antibody effector functions and half-life. Antibody effector functions, antibody-dependent cell-mediated cytotoxicity (ADCC) and complement-dependent cytotoxicity (CDC), are mediated by Fc residues in the lower hinge and nearby regions. Antibody half-life is dependent on recycling by binding to the neonatal Fc receptor (FcRn). The FcRn-binding region is also bound by Protein A during purification. In addition to affecting the efficacy and/or half-life of a product mutations or substitutions in or close to the Fc receptor regions may alter or purification possibilities of an Fc-containing product. Substitutions in the Fc are evaluated for their potential impact on purification and manufacturing.

In some embodiments, sequence variants detected by the methods described herein are further analysed to detect free cysteine thiol groups. Solvent exposed, free Cysteine thiol groups may cause problems such as protein misfolding, aggregation, non-specific tissue binding, increased immunogenicity through disulfide scrambling or unintended reactions with other molecules in the solution. A sequence search against an internal database is performed to locate related sequences and thereby conserved disulfide bonds. Cysteine residues that do not fit these conserved positions are considered liabilities. Structural analysis of these residues for their potential for disulfide formation and influence on folding and stability is also performed. Proteins, domains or linkers with known issues relating to disulfide bond are also detected. For example, human native IgG4 and IgG2 antibodies are susceptible to dissociation and hinge region disulfide scrambling, respectively.

In some embodiments, sequence variants detected by the methods described herein are further analysed to evaluate isoelectric point. The isoelectric point (pI) of a protein is the pH at which the protein has zero net electrical charge. When a protein solution is at a pH equal to the pI of the protein the repulsive electrostatic forces between charges on the protein molecules are minimised. The inadequate repulsion may increase the risk of hydrophobic surface patches becoming aggregation hot-spots. Local charge distribution across the molecules surface also influences the formulation design. The product's pI is evaluated to determine if the product will fit standard (antibody) purification processes (Liu et al 2010 MAbs). A more complex purification strategy should be pursued if the pI is far outside the standard range. The isoelectric point is calculated based on the number of charged residues in the primary amino-acid sequence using EMBOSS pKa values.

In some embodiments, sequence variants detected by the methods described herein are further analysed to detect lysine glycation. Glycation is a non-enzymatic modification that primarily affects the side-chain ε-amino group of Lysine. The modification commonly occurs during cell culturing when there is a high concentration of glucose. It is estimated that 5-20% of the recombinant proteins produced will have a glycated Lysine (Saleem et al. (2015). MAbs. 7. 719-731). All solvent exposed Lysines are potentially susceptible, however, negative charges and Histidine imidazole groups catalyse the modification and can cause an enrichment of Lysine glycation at susceptible sites. Lysine residues in critical regions with a Histidine or acidic residue side-chain within a catalytic distance of the Lysine side-chain ε-amino group are detected. This catalytic distance could be for example 5 Å, 10 Å or 20 Å.

In some embodiments, sequence variants detected by the methods described herein are further analysed to detect N- and O-glycosylation. Glycosylation is a common post-translational modification appearing in therapeutic proteins such as antibodies, blood factors, EPO, hormones and interferons (Walsh. (2010). Drug Discov. Today. 15. 773-780). Proper glycosylation is important not only for folding but also stability, solubility, potency, pharmacokinetics and immunogenicity. Unintended glycan structures in or near binding interfaces may sterically hinder binding and impact affinity. For N-glycosylation, the N-X-S/T motif where X is any residue except Proline generally serves to detect sites. However, not all such motifs are N-glycosylated and over a thousand other unique sites that do not conform to this motif are known. O-glycosylation of Serine and Threonine does not follow any simple pattern and a boosting decision tree ensemble algorithm was trained on experimentally determined glycosylation sites in order to predict O-glycosylation.

In some embodiments, sequence variants detected by the methods described herein are further analysed to detect N-terminal cyclisation. N-terminal cyclisation of a protein can occur through the nucleophilic attack of the N-terminal amine on the second carbonyl group of the backbone, producing diketopiperazine (DKP) (Liu et al. (2011). J. Biol. Chem. 286. 11211-11217). N-terminal cyclisation causes mass and charge heterogeneity which has to be controlled and monitored.

In some embodiments, sequence variants detected by the methods described herein are further analysed to detect oxidation. Several amino-acids are susceptible to damage by oxidation caused by reactive oxygen species (ROS). Histidine, Methionine, Cysteine, Tyrosine and Tryptophan are amongst them. Oxidation is generally divided into two categories: site-specific metal-catalysed oxidation and non-specific oxidation. Methionine and to a lesser extent Tryptophan are more susceptible to non-site specific oxidation. While Methionine is primarily sensitive to free ROS, Tryptophan is more sensitive to light-induced oxidation. The degree of sensitivity is determined in part by the solvent accessibility of the side-chain; buried residues are less sensitive or take longer to react. Structural analysis is used to determine at risk residues.

In some embodiments, sequence variants detected by the methods described herein are further analysed to detect pyroglutamate formation. Pyroglutamate formation is a modification occurring in proteins with an N-terminal Glutamine or Glutamic acid residue, where the side-chain cyclises with the N-terminal amine group to form a five-membered ring structure. N-terminal cyclisation causes mass and charge heterogeneity which has to be controlled and monitored (Liu et al. (2008). J. Pharm. Sci. 97. 2426-2447). Pyroglutamate formation is commonly found in antibodies with an N-terminal Glutamine. Pyroglutamate formation from N-terminal Glutamic acid can occur during manufacturing and has been found in vivo (Cai et al. (2011). Biotechnol. Bioeng. 108. 404-412). N-terminal Glutamine or Glutamic acid residues are detected.

In some embodiments, sequence variants detected by the methods described herein are further analysed to detect, predict, and/or evaluate one or more (e.g., 1, 2, 3, 4, 5, 6, 7, 8, 9, 10, 11, 12, or all) of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

### Denaturing Methods

In some embodiments, the methods and systems of the disclosure comprise denaturing purified protein products. Denaturing methods include heating, addition of chaotropic agents (e.g., guanidine HCl or urea alone), or addition of detergents (e.g., sodium dodecylsulphate, SDS).

In some embodiments, the methods and systems of the disclosure comprise denaturing a purified protein products using deoxycholate. Deoxycholate is stabilised in aqueous solution by the presence of urea (and without urea present precipitates out of solutions that contain substantial levels of salt). Both of these substances act to denature the analyte protein, allowing lower temperatures and incubation times to be used in sample preparation steps compared to alternative sample preparation methods. The gentle sample preparation conditions allowed by this method minimise modifications to the protein that can be induced by other preparation methods. At the end of the procedure, deoxycholate can be precipitated out of solution by addition of acid, while the analyte peptides (products of digestion) are stabilised in solution by the urea, resulting in a method that is compatible with analysis by mass spectrometry, (unlike most methods that include use of a detergent molecule).

The combination of both of these substances within the same sample preparation procedure is important for the effectiveness of the purified protein preparation procedure. Specifically, the stabilising interaction of urea with deoxycholate in high salt solutions and the stabilising interaction of urea with the analyte protein/peptides, e.g., the purified protein product, on removal of deoxycholate by acid precipitation is important to the methods disclosed herein.

### Applications for Production

The methods of preparation of products, e.g., product variants, disclosed herein can be used to produce a variety of products, evaluate various cell lines, or to evaluate the production of various cell lines for use in a bioreactor or processing vessel or tank, or, more generally with any feed source. The devices, facilities and methods described herein are suitable for culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products—such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermentor or fermentation unit, or any other reaction vessel and the term “reactor” is used interchangeably with “fermentor.” For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

In embodiments and unless stated otherwise herein, the devices, facilities, and methods described herein for use with methods of making a preparation can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

Methods of making a preparation described herein can use a broad spectrum of cells. In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBvl3.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57BI/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, N.C., USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS 115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12 Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the polypeptide is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide, calcitonin, etanercept, hemoglobin glutamer 250 (bovine), drotrecogin alpha, collagenase, carperitide, recombinant human epidermal growth factor, DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (Hydron), deslorelin, histrelin, nafarelin, leuprolide (ATRIGEL), leuprolide (DUROS), goserelin, Eutropin, somatropin, mecasermin, enlfavirtide, Org-33408, insulin glargine, insulin glulisine, insulin (inhaled), insulin lispro, insulin deternir, insulin (RapidMist), mecasermin rinfabate, anakinra, celmoleukin, 99 mTc-apcitide, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, dornase alpha, trafermin, ziconotide, taltirelin, diboterminalfa, atosiban, becaplermin, eptifibatide, Zemaira, CTC-111, Shanvac-B, octreotide, lanreotide, ancestirn, agalsidase beta, agalsidase alpha, laronidase, prezatide copper acetate, rasburicase, ranibizumab, Actimmune, PEG-Intron, Tricomin, recombinant human parathyroid hormone (PTH) 1-84, epoetin delta, transgenic antithrombin III, Granditropin, Vitrase, recombinant insulin, interferon-alpha, GEM-21S, vapreotide, idursulfase, omnapatrilat, recombinant serum albumin, certolizumab pegol, glucarpidase, human recombinant C1 esterase inhibitor, lanoteplase, recombinant human growth hormone, enfuvirtide, VGV-1, interferon (alpha), lucinactant, aviptadil, icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide, tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone, recombinant G-CSF, insulin, insulin (Technosphere), insulin (AERx), RGN-303, DiaPep277, interferon beta, interferon alpha-n3, belatacept, transdermal insulin patches, AMG-531, MBP-8298, Xerecept, opebacan, AIDSVAX, GV-1001, LymphoScan, ranpirnase, Lipoxysan, lusupultide, MP52, sipuleucel-T, CTP-37, Insegia, vitespen, human thrombin, thrombin, TransMID, alfimeprase, Puricase, terlipressin, EUR-1008M, recombinant FGF-I, BDM-E, rotigaptide, ETC-216, P-113, MBI-594AN, duramycin, SCV-07, OPI-45, Endostatin, Angiostatin, ABT-510, Bowman Birk Inhibitor, XMP-629, 99 mTc-Hynic-Annexin V, kahalalide F, CTCE-9908, teverelix, ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, SB-249553, Oncovax-CL, OncoVax-P, BLP-25, CerVax-16, MART-1, gp100, tyrosinase, nemifitide, rAAT, CGRP, pegsunercept, thymosinbeta4, plitidepsin, GTP-200, ramoplanin, GRASPA, OBI-1, AC-100, salmon calcitonin (eligen), examorelin, capromorelin, Cardeva, velafermin, 131I-TM-601, KK-220, T-10, ularitide, depelestat, hematide, Chrysalin, rNAPc2, recombinant Factor V111 (PEGylated liposomal), bFGF, PEGylated recombinant staphylokinase variant, V-10153, SonoLysis Prolyse, NeuroVax, CZEN-002, rGLP-1, BIM-51077, LY-548806, exenatide (controlled release, Medisorb), AVE-0010, GA-GCB, avorelin, ACM-9604, linaclotid eacetate, CETi-1, Hemospan, VAL, fast-acting insulin (injectable, Viadel), insulin (eligen), recombinant methionyl human leptin, pitrakinra, Multikine, RG-1068, MM-093, NBI-6024, AT-001, PI-0824, Org-39141, Cpn10, talactoferrin, rEV-131, rEV-131, recombinant human insulin, RPI-78M, oprelvekin, CYT-99007 CTLA4-Ig, DTY-001, valategrast, interferon alpha-n3, IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, ALTU-135, recombinant neuraminidase, Vacc-5q, Vacc-4x, Tat Toxoid, YSPSL, CHS-13340, PTH(1-34) (Novasome), Ostabolin-C, PTH analog, MBRI-93.02, MTB72F, MVA-Ag85A, FARA04, BA-210, recombinant plague FIV, AG-702, OxSODrol, rBetV1, Der-p1/Der-p2/Der-p7, PR1 peptide antigen, mutant ras vaccine, HPV-16 E7 lipopeptide vaccine, labyrinthin, WTI-peptide, IDD-5, CDX-110, Pentrys, Norelin, CytoFab, P-9808, VT-111, icrocaptide, telbermin, rupintrivir, reticulose, rGRF, HA, alpha-galactosidase A, ACE-011, ALTU-140, CGX-1160, angiotensin, D-4F, ETC-642, APP-018, rhMBL, SCV-07, DRF-7295, ABT-828, ErbB2-specific immunotoxin, DT3SSIL-3, TST-10088, PRO-1762, Combotox, cholecystokinin-B/gastrin-receptor binding peptides, 111In-hEGF, AE-37, trasnizumab-DM1, Antagonist G, IL-12, PM-02734, IMP-321, rhIGF-BP3, BLX-883, CUV-1647, L-19 based ra, Re-188-P-2045, AMG-386, DC/1540/KLH, VX-001, AVE-9633, AC-9301, NY-ESO-1 (peptides), NA17.A2 peptides, CBP-501, recombinant human lactoferrin, FX-06, AP-214, WAP-8294A, ACP-HIP, SUN-11031, peptide YY [3-36], FGLL, atacicept, BR3-Fc, BN-003, BA-058, human parathyroid hormone 1-34, F-18-CCR1, AT-1100, JPD-003, PTH(7-34) (Novasome), duramycin, CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix, EP-51216, hGH, OGP-I, sifuvirtide, TV4710, ALG-889, Org-41259, rhCC 10, F-991, thymopentin, r(m)CRP, hepatoselective insulin, subalin, L19-IL-2 fusion protein, elafin, NMK-150, ALTU-139, EN-122004, rhTPO, thrombopoietin receptor agonist, AL-108, AL-208, nerve growth factor antagonists, SLV-317, CGX-1007, INNO-105, teriparatide (eligen), GEM-OS 1, AC-162352, PRX-302, LFn-p24 fusion, EP-1043, gpE1, gpE2, MF-59, hPTH(1-34), 768974, SYN-101, PGN-0052, aviscumnine, BIM-23190, multi-epitope tyrosinase peptide, enkastim, APC-8024, GI-5005, ACC-001, TTS-CD3, vascular-targeted TNF, desmopressin, onercept, and TP-9201.

In some embodiments, the polypeptide is adalimumab (HUMIRA), infliximab (REMICADE™), rituximab (RITUXAN™/MAB THERA™) etanercept (ENBREL™), bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 of US2016/0097074:

TABLE 1

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion protein LFA3-Ig | Amevive ® |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta-glucocerebrosidase | Ceredase ® |
| imiglucerase; recombinant form of beta-glucocerebrosidase | Cerezyme ® |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor complex-human | Humate-P ® |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating factor | Leukine ®/Leukine ® Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| abatacept, fully human soluable fusion protein | Orencia ™ |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); gonadotropin-releasing hormone | Plenaxis ™ |
| antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell blocker | RAPTIVA ™ |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, humanized mAb | Synagis ™ |
| thyrotropin alfa | Thyrogen ® |
| Tenecteplase | TNKase ™ |
| Natalizumab | TYSABRI ® |
| human immune globulin intravenous 5% and 10% solutions | Venoglobulin-S ® |
| interferon alfa-n1, lymphoblastoid | Wellferon ® |
| drotrecogin alfa | Xigris ™ |
| Omalizumab; recombinant DNA-derived humanized monoclonal | Xolair ® |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molecule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, |
| | | Protropin, Siazen, Serostim, Valtropin |
| | Human follicle-stimulating | Gonal-F, Follistim |
| | hormone (FSH) | |
| | Human chorionic | Ovidrel |
| | gonadotropin | Luveris |
| | Lutropin-α | GlcaGen |
| | Glucagon | Geref |
| | Growth hormone releasing | ChiRhoStim (human peptide), SecreFlo |
| | hormone (GHRH) | (porcine peptide) |
| | Secretin | Thyrogen |
| | Thyroid stimulating | |
| | hormone (TSH), thyrotropin | |
| Blood | Factor VIIa | NovoSeven |
| Clotting/Coagulation | Factor VIII | Bioclate, Helixate, Kogenate, |
| Factors | | Recombinate, ReFacto |
| | Factor IX | Benefix |
| | Antithrombin III (AT-III) | Thrombate III |
| | Protein C concentrate | Ceprotin |
| Cytokine/Growth | Type I alpha-interferon | Infergen |
| factor | Interferon-αn3 (IFNαn3) | Alferon N |
| | Interferon-β1a (rIFN-β) | Avonex, Rebif |
| | Interferon-β1b (rIFN-β) | Betaseron |
| | Interferon-γ1b (IFN γ) | Actimmune |
| | Aldesleukin (interleukin | Proleukin |
| | 2 (IL2), epidermal | |
| | theymocyte activating | |
| | factor; ETAF | |
| | Palifermin (keratinocyte | Kepivance |
| | growth factor; KGF) | |
| | Becaplemin (platelet- | Regranex |
| | derived growth factor; | |
| | PDGF) | |
| | Anakinra (recombinant IL1 | Anril, Kineret |
| | antagonist) | |
| Antibody molecules | Bevacizumab (VEGFA | Avastin |
| | mAb) | |
| | Cetuximab (EGFR mAb) | Erbitux |
| | Panitumumab (EGFR mAb) | Vectibix |
| | Alemtuzumab (CD52 mAb) | Campath |
| | Rituximab (CD20 chimeric | Rituxan |
| | Ab) | |
| | Trastuzumab (HER2/Neu | Herceptin |
| | mAb) | |
| | Abatacept (CTLA Ab/Fc | Orencia |
| | fusion) | |
| | Adalimumab (TNFα mAb) | Humira |
| | Etanercept (TNF | Enbrel |
| | receptor/Fc fusion) | |
| | Infliximab (TNFα chimeric | Remicade |
| | mAb) | |
| | Alefacept (CD2 fusion | Amevive |
| | protein) | |
| | Efalizumab (CD11a mAb) | Raptiva |
| | Natalizumab (integrin α4 | Tysabri |
| | subunit mAb) | |
| | Eculizumab (C5mAb) | Soliris |
| | Muromonab-CD3 | Orthoclone, OKT3 |
| Other: | Insulin | Humulin, Novolin |
| Fusion | Hepatitis B surface antigen | Engerix, Recombivax HB |
| proteins/Protein | (HBsAg) | |
| vaccines/Peptides | HPV vaccine | Gardasil |
| | OspA | LYMErix |
| | Anti-Rhesus (Rh) | Rhophylac |
| | immunoglobulin G | |
| | Enfuvirtide | Fuzeon |
| | Spider silk, e.g., fibrion | QMONOS |

In embodiments, the protein is a multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant ascites |
| (Removab ®, | Triomab | EpCAM | cells to tumor, Fc | EU | in EpCAM |
| Fresenius Biotech, | | | mediated effector | | positive tumors |
| Trion Pharma, | | | functions | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 (AMG | BiTE | CD3, CEA | Retargeting of T | Phase I | Gastrointestinal |
| 211, MedImmune, | | | cells to tumor | | adenocancinoma |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, Bayer; | | | cells to tumor | | |
| Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal cancer |
| (Macrogenics) | | | cells to tumor | | |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of NK | | |
| Therapeutics) | | | cells to tumor | | |
| | | | cells | | |
| AFM13 (Affimed | TandAb | CD30, | Retargeting of NK | Phase II | Hodgkin's |
| Therapeutics) | | CD16A | cells to tumor | | Lymphoma |
| | | | cells | | |
| GD2 (Barbara Ann | T cells | CD3, GD2 | Retargeting of T | Phase I/II | Neuroblastoma |
| Karmanos Cancer | preloaded | | cells to tumor | | and |
| Institute) | with BsAb | | | | osteosarcoma |
| pGD2 (Barbara | T cells | CD3, Her2 | Retargeting of T | Phase II | Metastatic breast |
| Ann Karmanos | preloaded | | cells to tumor | | cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR-armed | T cells | CD3, EGFR | Autologous | Phase I | Colon and |
| activated T-cells | preloaded | | activated T cells | | pancreatic |
| (Barbara Ann | with BsAb | | to EGFR-positive | | cancers |
| Karmanos Cancer | | | tumor | | |
| Institute) | | | | | |
| rM28 (University | Tandem | CD28, | Retargeting of T | Phase II | Metastatic |
| Hospital Tübingen) | scFv | MAPG | cells to tumor | | melanoma |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University of | linked to | | protein toxin to | | or lymphoma |
| Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, Roche) | | | | | Colorectal cancer |
| LY3164530 (Eli | Not | EGFR, MET | Blockade of 2 | Phase I | Advanced or |
| Lily) | disclosed | | receptors | | metastatic cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, VEGF A | Blockade of 2 | Phase I | Solid tumors |
| (RO5520985, | | | proangiogenics | | |
| Roche) | | | | | |
| RG7716 (Roche) | CrossMab | Ang2, VEGF A | Blockade of 2 | Phase I | Wet AMD |
| | | | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL-17A | Blockade of 2 | Phase II | Rheumatoid |
| (AbbVie) | | | proinflammatory | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem IgG | | cytokines | | fibrosis |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, Ablynx) | | | proinflammatory | | arthritis |
| | | | cytokine, binds to | | |
| | | | HSA to increase | | |
| | | | half-life | | |
| ALX-0761 (Merck | Nanobody | IL-17A/F, | Blockade of 2 | Phase I | (Healthy |
| Serono, Ablynx) | | HSA | proinflammatory | | volunteers) |
| | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, Ablynx; | | | proinflammatory | | arthritis |
| | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

TABLE 4

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| Eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating | Leukine ®/Leukine ® |
| factor | Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone | |
| Antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell | RAPTIVA ™ |
| blocker | |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, | Synagis ™ |
| humanized mAb | |
| thyrotropin alfa | Thyrogen ® |
| Tenecteplase | TNKase ™ |
| Natalizumab | TYSABRI ® |
| human immune globulin intravenous 5% and | Venoglobulin-S ® |
| 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In some embodiments, the polypeptide is an antigen expressed by a cancer cell. In some embodiments the recombinant or therapeutic polypeptide is a tumor-associated antigen or a tumor-specific antigen. In some embodiments, the recombinant or therapeutic polypeptide is selected from HER2, CD20, 9-O-acetyl-GD3, βhCG, A33 antigen, CA19-9 marker, CA-125 marker, calreticulin, carboanhydrase IX (MN/CA IX), CCR5, CCR8, CD19, CD22, CD25, CD27, CD30, CD33, CD38, CD44v6, CD63, CD70, CC123, CD138, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5_{AC}, MUC5_{B}, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-alpha precursor, and combinations thereof.

In some embodiments, the polypeptide is an activating receptor and is selected from 2B4 (CD244), α₄β₁ integrin, β₂ integrins, CD2, CD16, CD27, CD38, CD96, CD100, CD160, CD137, CEACAM1 (CD66), CRTAM, CSI (CD319), DNAM-1 (CD226), GITR (TNFRSF18), activating forms of KIR, NKG2C, NKG2D, NKG2E, one or more natural cytotoxicity receptors, NTB-A, PEN-5, and combinations thereof, optionally wherein the β₂ integrins comprise CD11a-CD18, CD11 b-CD 18, or CD11c-CD 18, optionally wherein the activating forms of KIR comprise K1R2DS1, KIR2DS4, or KIR-S, and optionally wherein the natural cytotoxicity receptors comprise NKp30, NKp44, NKp46, or NKp80.

In some embodiments, the polypeptide is an inhibitory receptor and is selected from KIR, ILT2/LIR-1/CD85j, inhibitory forms of KIR, KLRG1, LAIR-1, NKG2A, NKR-P1A, Siglec-3, Siglec-7, Siglec-9, and combinations thereof, optionally wherein the inhibitory forms of KIR comprise KIR2DL1, KIR2DL2, KIR2DL3, KIR3DL1, KIR3DL2, or KIR-L.

In some embodiments, the polypeptide is an activating receptor and is selected from CD3, CD2 (LFA2, OX34), CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Ly108, NTBA, SLAMF6), CRTAM (CD355), DR3 (TNFRSF25), GITR (CD357), HVEM (CD270), ICOS, LIGHT, LTβR (TNFRSF3), OX40 (CD134), NKG2D, SLAM (CD150, SLAMF), TCRα, TCRβ, TCRδγ, TIM1 (HAVCR, KIM1), and combinations thereof.

In some embodiments, the polypeptide is an inhibitory receptor and is selected from PD-1 (CD279), 2B4 (CD244, SLAMF4), B71 (CD80), B7H1 (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1H (VISTA), TIGIT (VSIG9, VSTM3), TIM2 (TIMD2), TIM3 (HAVCR2, KIM3), and combinations thereof.

Other exemplary proteins include, but are not limited to any protein described in Tables 1-10 of Leader et al., “Protein therapeutics: a summary and pharmacological classification”, Nature Reviews Drug Discovery, 2008, 7:21-39 (incorporated herein by reference); or any conjugate, variant, analog, or functional fragment of the recombinant polypeptides described herein.

Other recombinant protein products include non-antibody scaffolds or alternative protein scaffolds, such as, but not limited to: DARPins, affibodies and adnectins. Such non-antibody scaffolds or alternative protein scaffolds can be engineered to recognize or bind to one or two, or more, e.g., 1, 2, 3, 4, or 5 or more, different targets or antigens.

In one embodiment, the vector comprising a nucleic acid sequence encoding a product, e.g., a polypeptide, e.g, a recombinant polypeptide, described herein further comprises a nucleic acid sequence that encodes a selection marker. In one embodiment, the selectable marker comprises glutamine synthetase (GS); dihydrofolate reductase (DHFR) e.g., an enzyme which confers resistance to methotrexate (MTX); proline, or an antibiotic marker, e.g., an enzyme that confers resistance to an antibiotic such as: hygromycin, neomycin (G418), zeocin, puromycin, or blasticidin. In another embodiment, the selection marker comprises or is compatible with the Selexis selection system (e.g., SUREtechnology Platform™ and Selexis Genetic Elements™, commercially available from Selexis SA) or the Catalant selection system.

In one embodiment, the vector comprising a nucleic acid sequence encoding a recombinant product described herein comprises a selection marker that is useful in identifying a cell or cells comprise the nucleic acid encoding a recombinant product described herein. In another embodiment, the selection marker is useful in identifying a cell or cells that comprise the integration of the nucleic acid sequence encoding the recombinant product into the genome, as described herein. The identification of a cell or cells that have integrated the nucleic acid sequence encoding the recombinant protein can be useful for the selection and engineering of a cell or cell line that stably expresses the product.

### Numbered Embodiments

The present invention may be defined in any of the following numbered paragraphs.

1. A method of analysing a plurality of cells, a method using the plurality of cells, or a polypeptide made by the plurality of cells, comprising:

a) culturing a plurality of cells, at least one cell of the plurality of cells comprising a nucleic acid sequence encoding a product comprising a first amino acid sequence, e.g., a production sequence, to make conditioned media comprising product;

b) subjecting a first sample of polypeptide from the conditioned media comprising product to a first sequence-based reaction, e.g., digestion with a proteolytic enzyme, to provide a first reaction product, e.g., a proteolytic fragment (and, optionally, e.g., subjecting the reaction product to a separation step, e.g., by mass spec);

c) comparing a value for the first reaction product, e.g., presence, mobility (e.g., time of flight) or molecular weight, with a reference value, e.g., a value for a reaction product produced by application of the first sequence-based reaction to a reference sequence, e.g., the first amino acid sequence, and responsive to the comparison, selecting a reaction product component for further analysis, e.g., sequencing;

d) subjecting a second sample of polypeptide from the conditioned media comprising product to a second sequence-based reaction, e.g., digestion with a second proteolytic enzyme, to provide a second reaction product, e.g., a proteolytic fragment (and, optionally, e.g., subjecting the reaction product to a separation step, e.g., by mass spec);

e) comparing a value for the second reaction product, e.g., presence, mobility (e.g., time of flight) or molecular weight, with a reference value, e.g., a value for a reaction product produced by application of the second sequence-based reaction to a reference sequence, e.g., the first amino acid sequence, and responsive to the comparison, selecting a reaction product component for further analysis, e.g., sequencing;

f) optionally, subjecting a third sample of polypeptide from the conditioned media comprising product to a third sequence-based reaction, e.g., digestion with a proteolytic enzyme, to provide a third reaction product, e.g., a proteolytic fragment (and, optionally, e.g., subjecting the reaction product to a separation step, e.g., by mass spec);

g) optionally, comparing a value for the third reaction product, e.g., presence, mobility (e.g., time of flight) or molecular weight, with a reference value, e.g., a value for a reaction product produced by application of the third sequence-based reaction to a reference sequence, e.g., the first sequence, and responsive to the comparison, selecting a reaction product component for further analysis, e.g., sequencing,

h) optionally, responsive to the results of c) and optionally e) and/or g), determining if a sequence other than the first amino acid sequence is present in the plurality of cells, thereby analysing a plurality of cells, a method using the plurality of cells, or a polypeptide made by the plurality of cells.

2. The method of paragraph 1, comprising further culturing the plurality of cells to make second conditioned media comprising product; and performing steps b-h on the second conditioned media.

3. The method of paragraph 2, comprising further culturing the plurality of cells to make third conditioned media comprising product; and performing steps b-h on the third conditioned media.

4. The method of paragraph 3, comprises further culturing the plurality of cells to make a subsequent, e.g., N^{th}, wherein N=4, 5, 6, 7, 8, 9, 10, 11, 12, 13, 14, 15, 16, 17, 18, 19, or 20, conditioned media comprising product; and performing steps b-h on the subsequent, e.g., N^{th} conditioned media.

5. The method of any of paragraphs 1-4, wherein the plurality of conditioned culture media, the conditioned culture media, or a second, third, or subsequent, e.g., N^{th} conditioned culture media are produced at different stages of the production of the product, e.g., at early, middle, or late stage of growth of the product producing culture, or at different cell line production stages.

6. The method of any of paragraphs 1-4, wherein the plurality of conditioned culture media, the conditioned culture media, or a second, third, or subsequent, e.g., N^{th} conditioned culture media are produced at different time points in the culturing of the plurality of cells (e.g., at 1, 2, 3, 4, 5, 6, 7, 8, 9, 10, 11, 12, 13, 14, 15, 16, 17, 18, 19, 20, 21, 22, 23, or 24 hour time points, or at 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day time points).

7. The method of any of paragraphs 1-6, comprising comparing:

i) the determination made in h) for one of a conditioned culture media, the conditioned culture media, or a second, third, or subsequent, e.g., N^{th} conditioned culture media, with

ii) the determination made in h) for another of a conditioned culture media, the conditioned culture media, or a second, third, or subsequent, e.g., N^{th} conditioned culture media.

8. The method of paragraph 1, further comprising analyzing a second plurality of cells, comprising performing steps a-h on the second plurality of cells.

9. The method of paragraph 8, further comprising analyzing a third plurality of cells, comprising performing steps a-h on the third plurality of cells.

10. The method of paragraph 9, further comprising analyzing a subsequent, e.g., N^{th}, wherein N=4, 5, 6, 7, 8, 9, 10, 11, 12, 13, 14, 15, 16, 17, 18, 19, or 20, plurality of cells, comprising performing steps a-h on the subsequent, e.g., N^{th}, plurality of cells.

11. The method of any of paragraphs 8-10, comprising comparing:

i) the determination made in h) for one of the plurality of cells, the second, third, or subsequent, e.g., N^{th} plurality of cells, with

ii) the determination made in h) for another of the plurality of cells, second, third, or subsequent, e.g., N^{th} plurality of cells.

12. The method of any of paragraphs 8-11, wherein each plurality of cells comprises cells of the same type, e.g., the same species, the same cell line (e.g., CHO, NSO, HEK), or the same isolate of a cell line (e.g., the same isolate of a CHO cell line).

13. The method of any of paragraphs 8-11, wherein one or more, e.g., each, of the plurality of cells comprises cells of a different type, e.g., different species, different cell lines (e.g., CHO, NSO, HEK), or different isolates of a cell line (e.g., different isolates of a CHO cell line).

14. The method of any of paragraphs 11-13, comprising, responsive to the comparison, selecting a plurality of cells, e.g., for producing a product comprising the first amino acid sequence, e.g. a plurality of cells that does not comprise product comprising a sequence other than the first amino acid sequence.

15. The method of any of paragraphs 1-14, wherein the first amino acid sequence corresponds to a protein product selected from Tables 1-4.

16. The method of any of paragraphs 1-15, wherein b), d), and optionally f) comprise denaturing the sample of polypeptide.

17. The method of paragraph 16, wherein denaturing the purified protein comprises incubating the purified protein in the presence of guanidine hydrochloride (GuHC1) and at an acidic pH (e.g., a pH of 6.8, 6.5, 6.3, 6, 5.8, or 5.5).

18. The method of paragraph 16, wherein denaturing the purified protein comprises incubating the purified protein in the presence of urea and deoxycholate.

19. The method of paragraph 18, wherein deoxycholate is precipitated out of solution prior to digestion of the purified protein product.

20. The method of paragraph 19, wherein the deoxycholate is precipitated out of solution prior to b), prior to d), and/or optionally prior to f).

21. The method of paragraph 19, wherein the deoxycholate is precipitated out of solution prior to optionally subjecting the reaction product to a separation step, e.g., by mass spec.

22. The method of any of paragraphs 19-21, wherein the deoxycholate is precipitated by the addition of an acid.

23. The method of any of paragraphs 1-22, wherein b), d), and/or optionally f) comprise reducing the purified protein with TCEP.

24. The method of any of paragraphs 1-22, wherein the sequence-based reaction is digestion with a proteolytic enzyme.

25. The method of paragraph 24, wherein the proteolytic enzyme is selected from trypsin, chymotrypsin, LysC, and AspN.

26. The method of any of paragraphs 1-25, wherein one or more steps is performed in an apparatus suitable for high throughput sample processing.

27. The method of paragraph 26, wherein one or more steps is performed in a 96-well plate.

28. The method of any of paragraphs 1-27, wherein b), d), and/or optionally f) optionally comprise a separation step comprising analyzing the reaction product, e.g., proteolytic fragment, using LC/MS.

29. The method of any of paragraphs 1-28, wherein c), e), and/or optionally g) comprise identifying the amino acid sequence of a component of the reaction product, e.g., a proteolytic fragment, identified by the comparison.

30. The method of paragraph 29, wherein identifying the amino acid sequence comprises using MS/MS on the component of the reaction product, e.g., a proteolytic fragment, identified by the comparison.

31. The method of any of paragraphs 1-30, wherein the method is automated.

32. The method of any of paragraphs 1-31, wherein the method employs robotic equipment.

33. The method of any of paragraphs 1-32, wherein the method employs a micro-fluidics system.

34. The method of any of paragraphs 1-33, further comprising, before b), d), and/or optionally f), purifying the product from the conditioned media containing product.

35. The method of paragraph 34, wherein purifying the product comprises using chromatography.

36. The method of any of paragraphs 1-35, comprising a washing protocol to remove carryover contamination from equipment.

37. The method of paragraph 36, wherein the washing protocol comprises analyzing a blank sample using LC/MS.

38. The method of paragraph 36, wherein the washing protocol comprises alternate washes of acidic solution and high organic solution.

39. The method of either paragraph 36 or 38, wherein the washing protocol can run in parallel to the method of analyzing a plurality of cells, a method using the plurality of cells, or a polypeptide made by the plurality of cells.

40. The method of paragraph 39, wherein the washing protocol does not add to the elapsed time of the method of analyzing a plurality of cells, a method using the plurality of cells, or a polypeptide made by the plurality of cells.

41. The method of paragraph 39, wherein running the washing protocol in parallel to the method of analyzing a plurality of cells, a method using the plurality of cells, or a polypeptide made by the plurality of cells reduces the elapsed time of the method by at least about 50%, 40%, 30%, 20%, or 10%.

42. The method of paragraph 39, wherein running the washing protocol in parallel to the method of analyzing a plurality of cells, a method using the plurality of cells, or a polypeptide made by the plurality of cells reduces additional time spent washing by at least about 90%, 80%, 70%, 60%, 50%, 40%, 30%, 20%, or 10%.

43. The method of any of paragraphs 1-42, further comprising evaluating the immunogenicity of the sequence other than the first amino acid sequence detected in part h).

44. The method of paragraph 43, wherein evaluating the immunogenicity comprises evaluating the sequence other than the first amino acid sequence detected in part h) using an in silico immunogenicity tool, e.g., Epibase.

45. A method of detecting a protein sequence variant, the method comprising:

a) providing a population of cells, wherein the cells produce a protein product;

b) purifying the protein product from the population of cells;

c) preparing the purified protein product for analysis by mass spectrometry;

d) analyzing the prepared purified protein product by mass spectrometry;

wherein a)-d) are repeated, in parallel or consequentially, for a plurality (e.g., more than one, e.g., two, three, four, five, six, seven, eight, nine, ten or more) of populations of cells; and

e) detecting protein sequence variants by comparing mass spectrometry data from the plurality of populations of cells and a database of mass spectrometry data,

thereby detecting the protein sequence variant.

46. The method of paragraph 45, wherein the populations of cells of the plurality are produced at different stages of the production of the product, e.g., at early, middle, or late stage of growth of a product producing culture, or at different cell line production stages.

47. The method of paragraph 45, wherein the populations of cells of the plurality are produced at different time points in the culturing of the plurality of cells (e.g., at 1, 2, 3, 4, 5, 6, 7, 8, 9, 10, 11, 12, 13, 14, 15, 16, 17, 18, 19, 20, 21, 22, 23, or 24 hour time points, or at 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day time points).

48. The method of paragraph 45, wherein each population of cells comprises cells of the same type, e.g., the same species, the same cell line (e.g., CHO, NSO, HEK), or the same isolate of a cell line (e.g., the same isolate of a CHO cell line).

49. The method of paragraph 45, wherein one or more, e.g., each, of the populations of cells comprises cells of a different type, e.g., different species, different cell lines (e.g., CHO, NSO, HEK), or different isolates of a cell line (e.g., different isolates of a CHO cell line).

50. The method of any of paragraphs 45-49, comprising, responsive to e), selecting a population of cells, e.g., for producing the product, e.g. a population of cells that does not comprise a protein sequence variant.

51. The method of any of paragraphs 45-50, wherein the protein product is a recombinant or therapeutic protein selected from Tables 1-4.

52. The method of any of paragraphs 45-51, wherein c) comprises denaturing the purified protein.

53. The method of paragraph 52, wherein denaturing the purified protein comprises incubating the purified protein in the presence of guanidine hydrochloride (GuHCl) and at an acidic pH (e.g., a pH of 6.8, 6.5, 6.3, 6, 5.8, or 5.5).

54. The method of paragraph 52, wherein denaturing the purified protein comprises incubating the purified protein in the presence of urea and deoxycholate.

55. The method of paragraph 54, wherein the deoxycholate is precipitated out of solution prior to digestion of the purified protein product.

56. The method of paragraph 54, wherein the deoxycholate is precipitated out of solution prior to d).

57. The method of any of paragraphs 54-56, wherein the deoxycholate is precipitated by the addition of an acid.

58. The method of any of paragraphs 45-57, wherein c) comprises reducing the purified protein with TCEP.

59. The method of any of paragraphs 45-58, wherein c) comprises digesting the purified protein with trypsin, chymotrypsin, LysC, or AspN.

60. The method of paragraph 59, wherein c) comprises forming a plurality of aliquots of the purified protein and digesting the aliquots with a plurality of proteases, wherein each aliquot is digested by a different protease, and wherein the protease is chosen from trypsin, chymotrypsin, LysC, or AspN.

61. The method of paragraph 60, wherein after digestion, the plurality of aliquots are mixed together.

62. The method of any of paragraphs 45-61, wherein c) is performed in an apparatus suitable for high throughput sample processing.

63. The method of paragraph 62, wherein c) is performed in a 96-well plate.

64. The method of any of paragraphs 45-63, wherein d) comprises analyzing the prepared purified protein product using LC/MS.

65. The method of any of paragraphs 45-64, wherein e) comprises identifying peptides displaying a change in abundance by comparative analysis of the data of the plurality of populations of cells and the mass spectrometry database.

66. The method of paragraph 65, wherein e) further comprises analyzing peptides displaying a change in abundance by MS/MS and identifying sequence alterations by comparing the MS/MS data to MS/MS databases.

67. The method of any of paragraphs 45-66, wherein the method is automated.

68. The method of any of paragraphs 45-67, wherein the method employs robotic equipment.

69. The method of any of paragraphs 45-68, wherein the method employs a micro-fluidics system.

70. The method of any of paragraphs 45-69, wherein b) comprises purifying the protein product using chromatography.

71. The method of any of paragraphs 45-70, wherein d) comprises a washing protocol to remove carryover contamination.

72. The method of paragraph 71, wherein the washing protocol comprises analyzing a blank sample using LC/MS.

73. The method of paragraph 71, wherein the washing protocol comprises alternate washes of acidic solution and high organic solution.

74. The method of either paragraph 71 or 73, wherein the washing protocol can run in parallel to the method of detecting a protein sequence variant.

75. The method of paragraph 74, wherein the washing protocol does not add to the elapsed time of the method of detecting a protein sequence variant.

76. The method of paragraph 74, wherein running the washing protocol in parallel to the method of detecting a protein sequence variant reduces the elapsed time of the method by at least about 50%, 40%, 30%, 20%, or 10%.

77. The method of paragraph 74, wherein running the washing protocol in parallel to the method of detecting a protein sequence variant reduces additional time spent washing by at least about 90%, 80%, 70%, 60%, 50%, 40%, 30%, 20%, or 10%.

78. The method of any of paragraphs 45-77, further comprising evaluating the immunogenicity of a detected protein sequence variant.

79. The method of paragraph 78, wherein evaluating the immunogenicity of a detected protein sequence variant comprises evaluating the protein sequence variant using an in silico immunogenicity tool, e.g., Epibase.

80. The method of any of paragraphs 1-44, wherein the method further comprises subjecting the first, second, and/or third samples of polypeptide to additional analysis to identify, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

81. The method of any of paragraphs 1-44, wherein, if a sequence other than the first amino acid sequence is present in the plurality of cells, subjecting the sequence other than the first amino acid sequence to additional analysis to identify, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

82. The method of any of paragraphs 45-79, wherein the method further comprises:

f) analysing the detected protein sequence variant(s) to identify, detect, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

83. A method of analysing a protein sequence variant as detected in any of paragraphs 45-79, wherein the method comprises one or more of the following: evaluating immunogenicity, predicting protein aggregation, e.g., propensity of protein aggregation; evaluating deamidation; detecting aspartic acid isomerisation and fragmentation; detecting C-terminal lysine processing; predicting/evaluating Fc ADCC/CDC response, half-life, and protein A purification; detecting free cysteine thiol groups; evaluating isoelectric point, detecting lysine glycation; identifying N- and/or O-glycosylation; detecting N-terminal cyclisation; detecting oxidation; or detecting pyroglutamate formation.

84. A method of analysing a sequence, e.g., a sequence other than a first sequence as identified in paragraphs 1-44, wherein the method comprises one or more of the following: evaluating immunogenicity, predicting protein aggregation, e.g., propensity of protein aggregation; evaluating deamidation; detecting aspartic acid isomerisation and fragmentation; detecting C-terminal lysine processing; predicting/evaluating Fc ADCC/CDC response, half-life, and protein A purification; detecting free cysteine thiol groups; evaluating isoelectric point, detecting lysine glycation; identifying N- and/or O-glycosylation; detecting N-terminal cyclisation; detecting oxidation; or detecting pyroglutamate formation.

85. A method of analysing a plurality of cells, the method comprising:

a) culturing a plurality of cells, at least one cell of the plurality of cells comprising a nucleic acid sequence encoding a product, said product comprising a first amino acid sequence, to make conditioned media comprising product;

b) subjecting a first sample of polypeptide from the conditioned media comprising product to a first sequence-based reaction to provide a first reaction product;

c) comparing a value for the first reaction product with a reference value, and responsive to the comparison, selecting a reaction product component for further analysis;

d) subjecting a second sample of polypeptide from the conditioned media comprising product to a second sequence-based reaction to provide a second reaction product;

e) comparing a value for the second reaction product with a reference value, and responsive to the comparison, selecting a reaction product component for further analysis;

f) optionally, subjecting a third sample of polypeptide from the conditioned media comprising product to a third sequence-based reaction to provide a third reaction product;

g) optionally, comparing a value for the third reaction product with a reference value, and responsive to the comparison, selecting a reaction product component for further analysis,

h) responsive to the results of c) and optionally e) and g), determining if a sequence other than the first amino acid sequence is present in the plurality of cells, thereby analysing a plurality of cells.

86. A method of detecting a protein sequence variant, the method comprising:

a) providing purified protein product from culture media comprising a population of cells, e.g., a plurality of cells, wherein the cells produce a protein product;

b) analyzing the purified protein product by mass spectrometry;

wherein a)-b) are repeated, in parallel or sequentially, for a plurality of samples within the same population of cells or different populations of cells; and

c) detecting protein sequence variants within the plurality of samples by comparing mass spectrometry data from the plurality of samples and a database of mass spectrometry data,

thereby detecting the protein sequence variant.

87. The method of any of the preceding paragraphs, wherein the sample is an aliquot.

88. A polypeptide made by the plurality of cells of the method of any of the preceding paragraphs.

### EXEMPLIFICATION

### Example 1: Introduction, Definitions, Parameters, Materials and Methods

Protein sequence variants are unintended amino acid sequence changes that can occur as a result of genomic nucleotide change or translational misincorporation. Systematic screening is emerging as an integral analytical component of cell line construction processes for successful manufacturing of biopharmaceuticals.

Understanding the propensity for expression systems to generate sequence variants enables an effective risk mitigation strategy. Interim misincorporation rates were examined for GS-CHO Xceed Expression System™. Mechanisms of misincorporation and correlation with cell line stability at early and late generation numbers were investigated. Method capabilities were considered with respect to the variability of the detection limit for sequence variants at different locations within an antibody product

TABLE 5
Analytical Target Profile (Continuation)

| Performance | | |
|---|---|---|
| Parameter | Target | Desired Target |
| Specificity | >100% redundant sequence | Maximise |
| | coverage | |
| LOD | 1% at any amino acid substitution in | Minimise |
| | a comparative screen | |

### Definitions

PSVA—Protein Sequence Variant Analysis
RP-LC-MS—Reverse Phase Liquid Chromatography Mass Spectrometry
LOD—Limit of Detection
DDA—Data Dependent Acquisition
MVA—Multivariate Analysis
PSM—Peptide-Spectrum Match
FDR—False Discovery Rate
ACN—Acetonitrile
FA—Formic Acid
TFA—Trifluoroacetic Acid
NL—Normalised Intensity Level
MS1—Mass Spectrometry Data (used for peptide mass fingerprinting)
MS2—Tandem Mass Spectrometry Data (data dependent fragmentation of a peptide by mass spectrometer)
LC-MS^{E}—Liquid Chromatography Mass Spectrometry with Data Independent Fragmentation
TIC—Total Ion Chromatogram
EIC—Extracted Ion Chromatogram
m/z—Mass-to-Charge Ratio
z—Charge
RCF—Relative Centrifugal Force

### Equipment:

Waters Xevo G2 QTOF mass spectrometer systems (Maintenance No 270420 and 271550)
Orbitrap Fusion mass spectrometer system (Maintenance No 309202 and 309203)

### Software:

MassLynx v4.1
BiopharmaLynx 1.2
Chromeleon 6.8
Tune 2.0
Progenesis QI
PEAKS Studio (Bioinformatics Solutions)

### Materials:

Rituximab batch B6026B01, 10 mg/ml, stored at −65° C. or below
Rituximab batch H0017B01, 10 mg/ml, stored at −65° C. or below
Herceptin batch 822601, 21 mg/ml, stored at −65° C. or below
Biosimilar candidate of transtuzumab batch P20504006, 4.7 mg/ml, stored at −65° C. or below
cB72.3 batch L22661/B10, 10.5 mg/ml, stored at −65° C. or below
cB72.3 cell culture supernatant (early and late phase) for cell lines 2A6, 1A12, 3C10, 3C12, 2F7, E22, stored −20 C
Biosimilar candidate of rituximab, batch 213976ARS, 49.3 mg/ml, stored at −65° C. or below
Site-specific Antibody Conjugate construct trastuzumab LC T180C, HC S160C and K217R, 0.57 mg/ml, stored at 5±3° C.
Site-specific Antibody Conjugate construct trastuzumab LC T180C, HC S160C and K217R, 18.7 mg/ml, stored at −65° C. or below

### Method:

The outline of the method is as follows:
Dilute protein samples to ≤10 mg/ml in water
Speedvac 0.12 mg sample aliquots to dryness
Redissolve each sample in 90 ul denaturation buffer
Incubate samples
Prepare one Zeba spin desalting column per sample
   Remove storage solution by centrifugation at 1500 g for 1 min.
   Add 300 μl digestion buffer to the top of the resin bed and centrifuge at 1500 g for 1 min.
   Repeat column equilibration twice.
   Apply the total volume of each sample to the centre of the compact resin bed. Centrifuge at 1500 g for 2 min, retain the column eluate.
To 75 μl of each sample, add 251 trypsin digestion solution per sample and blank and mix by vortex. Samples are incubated at 30±2° C. for 195±10 min.
Add 2 μl TFA to each sample and vortex
Centrifuge samples in a benchtop centrifuge for 10 min at 14,000 rcf.
Remove supernatant for analysis
Analyse by LC-MS

### Example 2: Sample Preparation

A workflow comprising of independent protein digestion with multiple enzymes and combining the inactivated digests before analysis was selected for protein sequence variant analysis.

Benefits of utilizing a separate multi-enzyme digest include:
Obtaining redundant protein sequence coverage with minimal method optimization for a given protein
Independent confirmation and quantitation of sequence variants using overlapping peptide sequences from each digest
Maximized sequence confirmation from peptide fragmentation utilizing fragmentation selectivity differences

Proteases evaluated in this study were: trypsin, lysC, chymotrypsin and aspN. Trypsin, chymotrypsin and aspN were selected for initial assessment due to the enzymes' complementary specificity. Optimization of digestion condition was performed for the selected proteases.

Samples are diluted to ≤10 mg/ml with MilliQ water. Replicate 0.12 mg aliquots of each diluted protein sample are placed on a 96-well plate in a randomized order. Samples are concentrated to dryness in a speedvac. 90 μl of denaturation buffer is added to each sample and the plate is incubated. Zeba Spin Desalting Plates, 96-well, 7K, are equilibrated with a urea-based digestion buffer as per manufacturer's instructions. The full volume of each denatured sample is transferred to the desalting plate and spun at 1000 g for 2 min. Aliquots of each sample are transferred to separate plates for a specific digestion (e.g. tryptic, chymotryptic, aspN, LysC). Digestion is performed at the specific enzyme to protease ratio, at a controlled time and temperature. The reaction is quenched by addition of 2% TFA.

### Optimization of Digestion

The following digestion attributes were determined to assess suitability of a digestion for PSVA:
Complete (no residual undigested peptides with more than 3 missed cleavages) Incomplete digestion affects quantitative capacity of an analysis.
Reproducible

Variation in the peptide map may affect comparative analysis and effective identification of sequence variants with Progenesis QI software.
Providing 100% sequence coverage

The effect of urea molarity and temperature on digestion were evaluated by assessing incomplete digestion, sequence coverage and peptide solubility. Antibody refolding can occur at low concentrations of urea (alongside possible peptide solubility issues) affecting the digestion efficiency.

Optimisation of digestion conditions was performed using three different molecules—rituximab, trastuzumab and cB72.3.

Digestion was performed overnight, using digestion buffer containing 0.1M tris-hydrochloride, urea as well as 1 mM TCEP to preserve reduced cysteines. The pH of the buffer was pH8 which falls in the pH range for optimal activity of each evaluated enzyme. The following conditions were assessed as part of the optimization process:
Urea molarity: 0.5M, 1M and 2M
Incubation temperature: 25° C., 30° C. and 37° C.

### Trypsin

Tryptic digestion was performed with varying urea molarity (0.5M, 1M and 2M) and temperature (25° C., 30° C. and 37° C.). Incubation was performed overnight and the enzyme to protein ratio was 1:20.

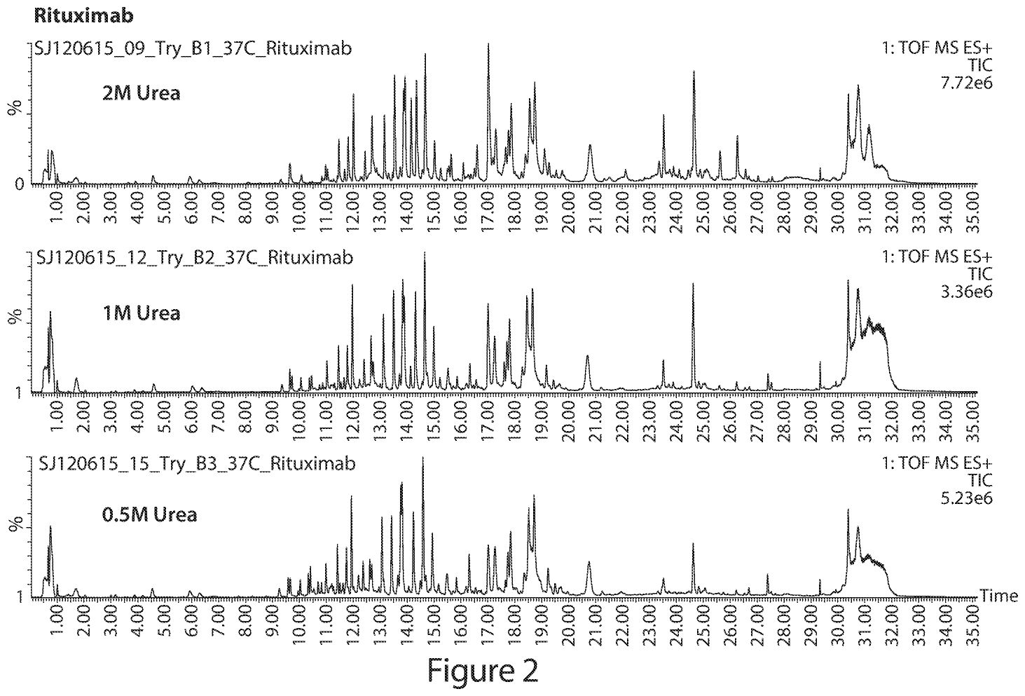

No evidence of undigested protein was observed on visual inspection of chromatograms (FIG. 2). Sequence coverage of 297% achieved for a single missed cleavage allowed, with only dipeptides not detected with the automated search.

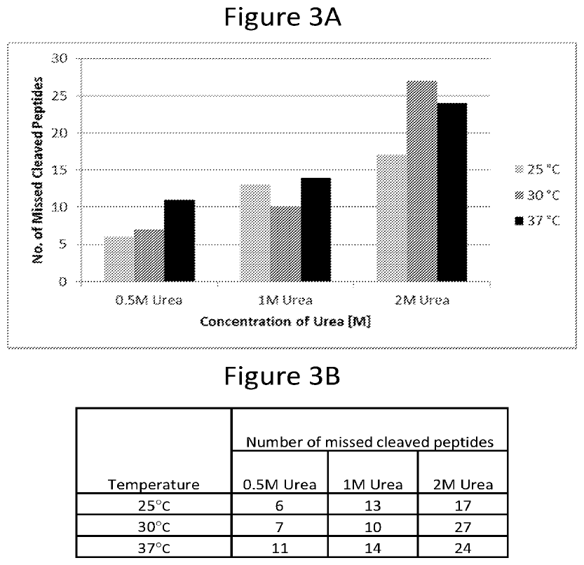

Efficiency of digestion was also evaluated by comparing the number of peptides generated by incomplete digestion (peptides with 1 missed cleavage or more). The smallest population of the missed cleavages was observed for digestion at 25° C. in 0.5M urea (FIG. 3).

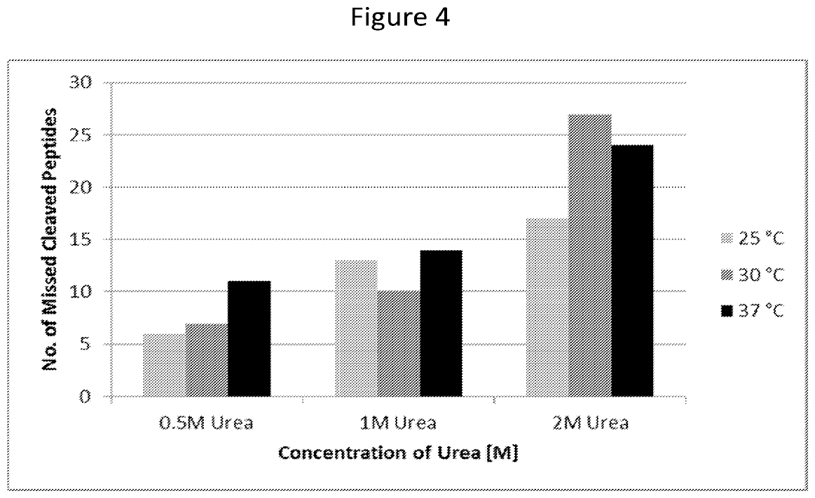

In addition, the he effect of urea molarity on the digestion was evaluated using the heavy chain peptide GFYPSDIAVEWESNGQPENNYK, which is known to be affected in the event of antibody refolding. Normalized intensity of the peptide was compared for all conditions (FIG. 4).

Reproducibility of the digestion for each condition was evaluated by visual examination of the chromatographic plots. Comparable chromatograms were observed for each condition.

### Chymotrypsin

Chymotryptic digestion was performed with varying urea molarity (0.5M, 1M and 2M) and temperature (25° C., 30° C. and 37° C.). Incubation was performed overnight and the enzyme to protein ratio was 1:20.

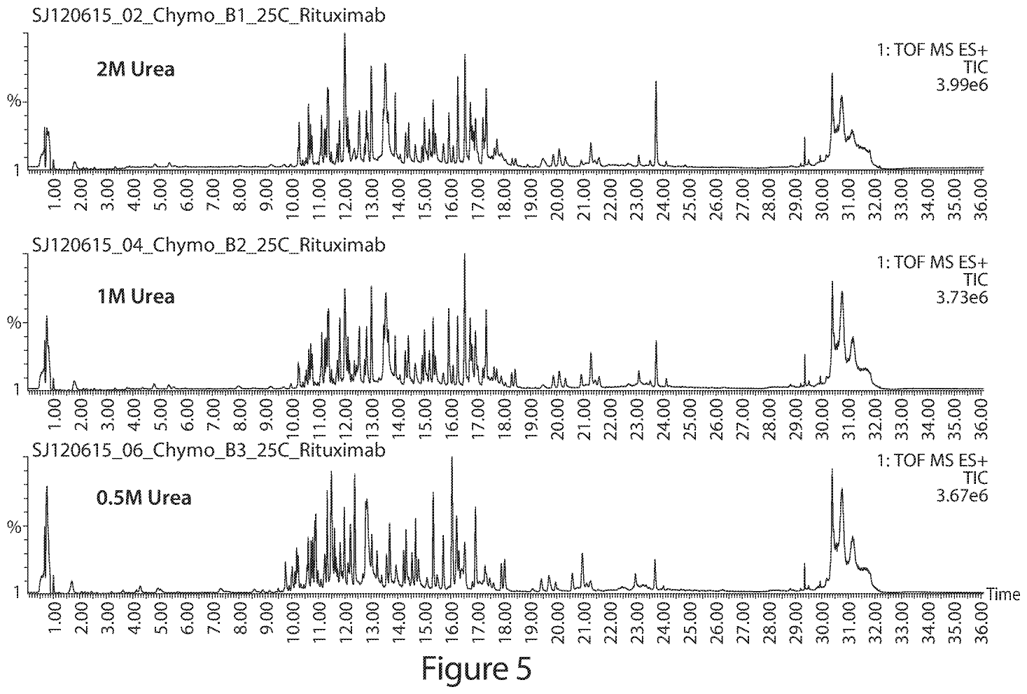

Satisfactory digestion efficiency was achieved for all evaluated conditions. No evidence of undigested protein was observed on visual inspection of chromatograms (FIG. 5). Sequence coverage of ≥95% achieved for a single missed cleavage allowed, with only dipeptides not covered in the automated search.

Efficiency of digestion was also evaluated by comparing intensity of a large heavy chain peptide generated from incomplete digestion of trastuzumab:

Y19-28
AMDYWGQGTLVTVSSASTKGPSVFPLAPSSKSTSGGTAALGCLVKDYFPEP
VTVSW.

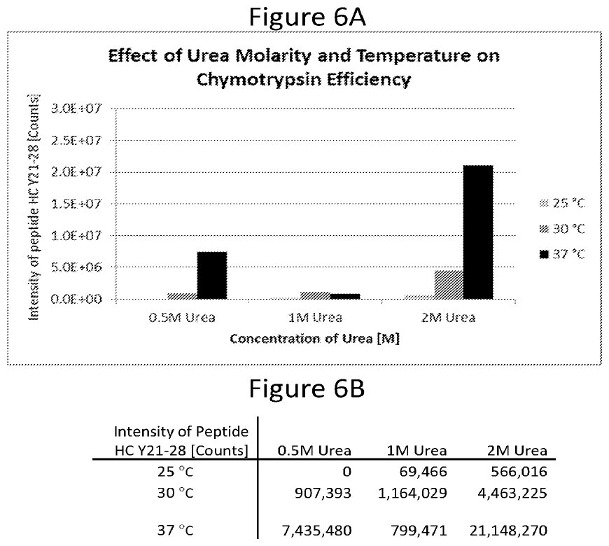

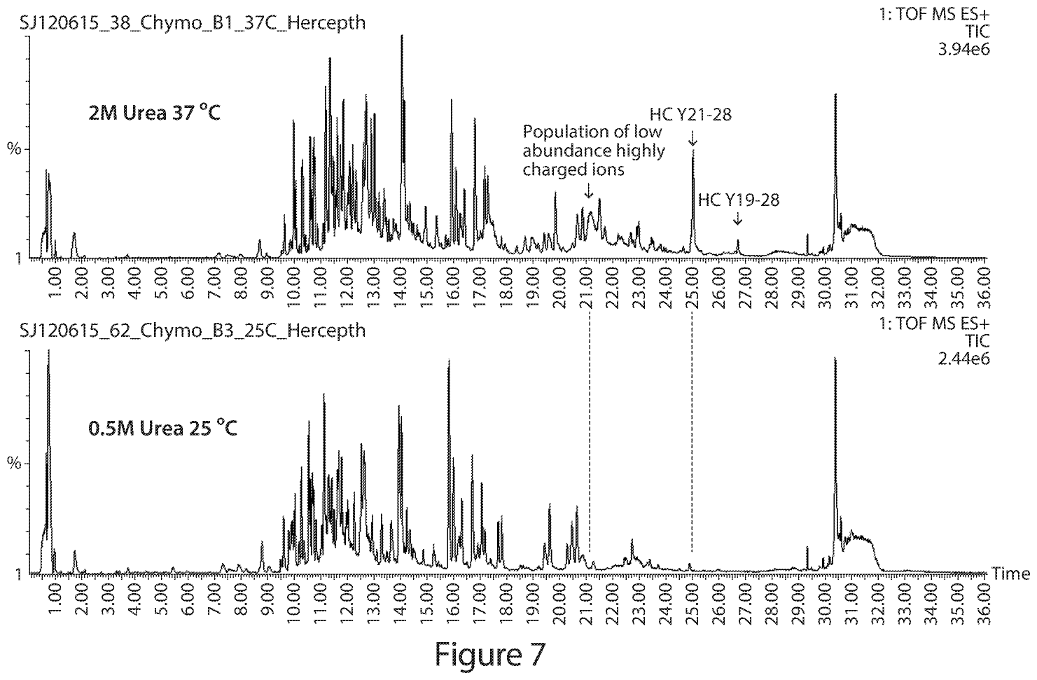

The lowest abundance of this mis-cleaved peptide (indicating more efficient digestion) was observed for conditions at 25° C. in 0.5M urea, as it was not detected (FIG. 6 and FIG. 7).

### AspN

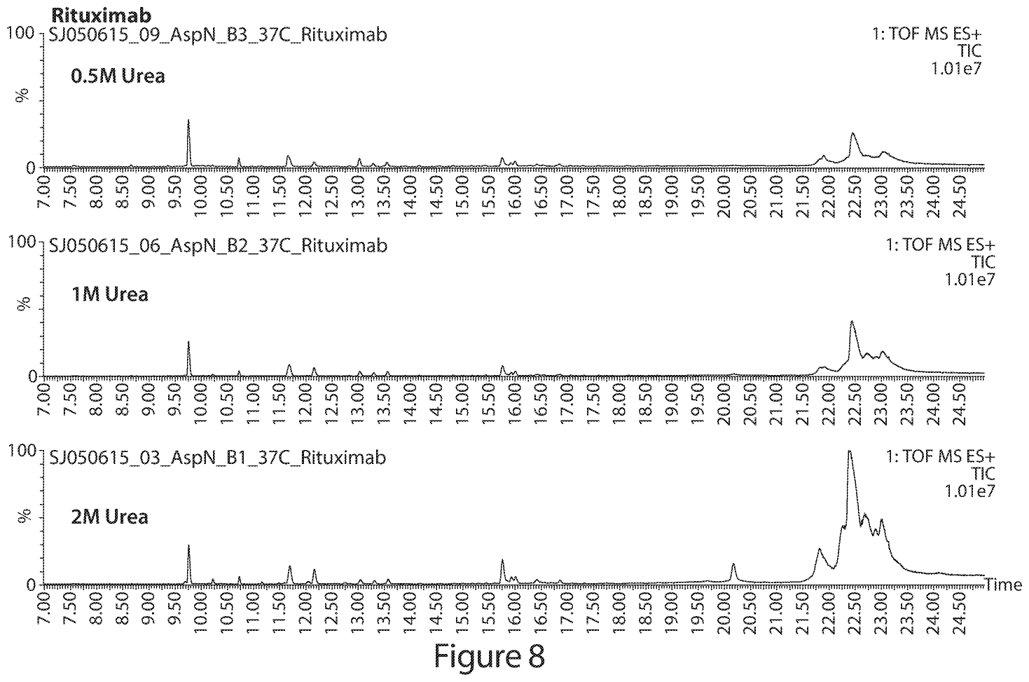

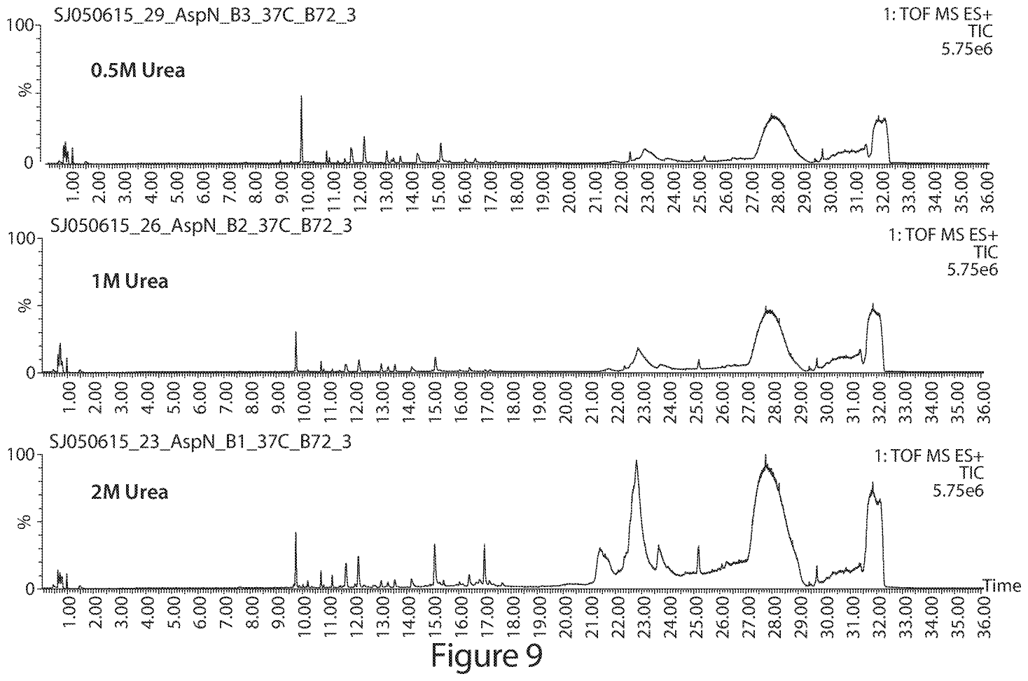

AspN digestion was performed with varying urea molarity (0.5M, 1M and 2M). Overnight incubation at 37° C. was performed using an enzyme to protein ratio of 1:40. Some evaporation of the samples was observed, due to the elevated temperature and long incubation time, which would have affected the composition of the digestion buffer. Large peaks representing undigested protein were detected, indicating that the digestion process was inefficient. The abundance of the undigested material was higher in 2M urea than in 1M or 0.5M urea (FIG. 8 and FIG. 9). Optimisation of the procedure would be required before AspN could be incorporated as one of the digestion enzymes for PSVA sample preparation. Further optimisation could be performed using a urea concentration below 1M and evaluating the effect of the addition of zinc acetate to increase the activity of Asp-N. It was decided not to take this forward as part of the digestion procedure at this time, and therefore was not included as part of the sample preparation procedure.

### Combined Digests

Tryptic/chymotryptic combined digest of trastuzumab sample was prepared by independent proteolysis with trypsin and chymotrypsin in 0.5M Urea. The incubation was performed overnight at 25° C. with an enzyme to protein ratio of 1:20. The digestion was quenched with 2% TFA and digests were combined. The sample was analysed using two LC-MS systems, utilising both standard and nano-flow configurations.

Complete sequence coverage was achieved for the combined tryptic and chymotryptic digest for RP-LC-MS1 analysis with Waters Xevo G2 QToF.

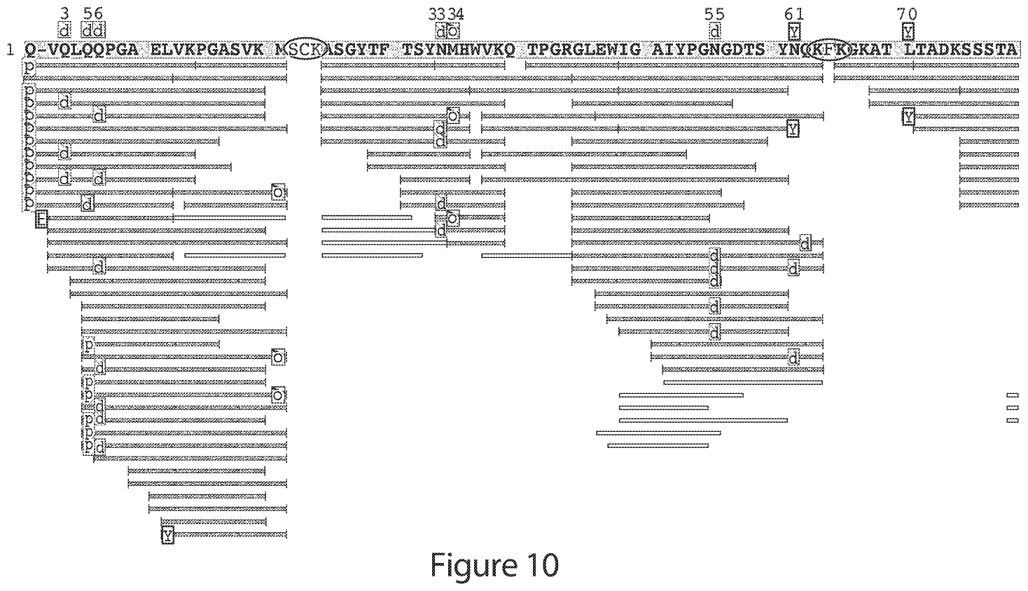

Incomplete sequence coverage was obtained for the combined tryptic and chymotryptic digest with nanoLC-MS2 analysis with Thermo Orbitrap Fusion. Analysis of trastuzumab digest with PEAKS Studio resulted in 100% MS2 coverage for the light chain and 99% coverage for the heavy chain. One tripeptide and one single residue peptide were not detected in the heavy chain (FIG. 10).

Application of nano-flow LC involves trapping the analyte on a C18 trapping column prior to the analytical column. Small peptides (usually below 5 amino acid residues) are not retained on this column. Likewise an appropriate peptide size is required for sequence confirmation with MS2 data.

Overnight digestion with trypsin and chymotrypsin generates small peptides which resulted in incomplete coverage for some regions of protein sequence. Furthermore, an extensive activity of chymotrypsin led to high cleavage rate at sites Met, Ala, Asp and Glu as well as some non-specific digestion in addition to expected digestion at Tyr, Phe, Trp and Leu. The current digestion workflow was determined to be unsuitable for nanoLC application.

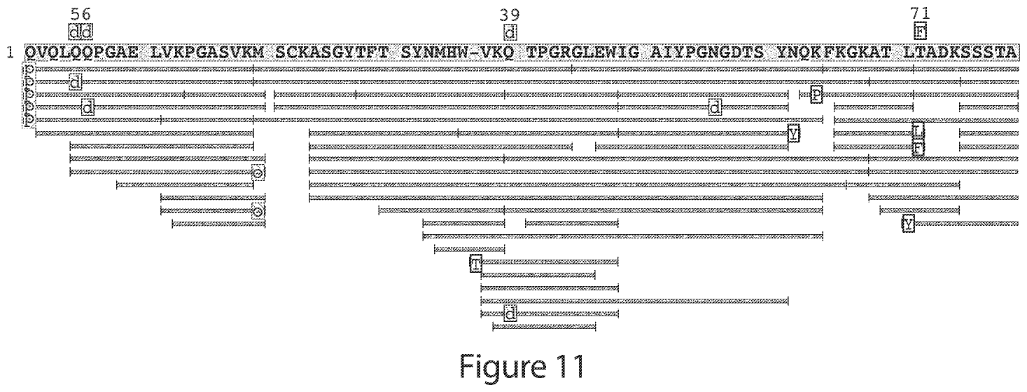

In order to generate an appropriate population of peptides for nano-flow configuration the digestion workflow was modified. LysC protease was introduced in addition to trypsin and chymotrypsin. Digestion time was reduced to 195 minutes to minimise non-specific digestion. 100% of the trastuzumab sequence was detected by PEAKS studio with the new digestion procedure (FIG. 11).

### Example 3: LC-MS Analysis

The principle of the protein sequence variant analysis (PSVA) is a comparative screening of protein peptide maps with application of multivariate analysis and an identification of the significantly different species with MS2 analysis.

Various factors require consideration to generate a successful method. PSVA is targeted at the cell line construction stage, and as such is required to be a robust, high throughput method.

Reproducible chromatography, comprehensive MS1 characterisation for each chromatographic peak and minimum sample carryover are important for the statistical analysis. PSVA is reliant on detection of variants at low levels, therefore sensitivity and a wide dynamic scan range are also important. Sequence coverage by MS2 depends on accurate and fast detection, with additional targeted fragmentation if required for identification of putative variants.

Regular low millilitre/min flow UHPLC as oppose to nano-flow LC has a benefit of high reproducibility and is less prone to carryover.

The model enables adaptation of separation technique depending on application using the output equations describing peak capacity, peak shape and sensitivity in relation to set LC parameters.

The model was used to develop a short LC method suitable for high-throughput protein sequence variant analysis. The method was recalculated to minimum gradient length required to meet defined quality parameter.

### Example 4: Method Outline

### Sample Preparation

Protein samples are diluted to ≤10 mg/ml in water
Duplicate aliquots of each sample are placed on the 96-well plate in a randomized order
Samples are concentrated to dryness in speedvac
90 μl of denaturation reduction buffer is added to each sample
Samples are incubated
Zeba Spin Desalting Plates, 96-well, 7K are prepared as per manufacturer instruction
   Plate is centrifuges at 1000×g for 2 minutes to remove the storage buffer. The flow-through is discarded.
   250 μL of digestion buffer is added on top of the resin bed. Centrifugation is performed at 1000×g for 2 minutes and the flow-through discarded. The step is performed 4 times in total.
   Total volume of each sample is added to the centre of the compact resin bed.
   Plate assembly is centrifuged at 1000×g for 2 minutes to collect processed sample.
Two 25 μl aliquots are taken for tryptic and chymotryptic digestion
All digestion are performed at 1:20 enzyme to protein ratio. 8.3 μl of 0.2 mg/ml enzyme is added to each set of aliquots for specific proteolysis.
Samples are incubated.
All digests are quenched separately with 2 μl of a one in three dilution of TFA.

### LC-MS Analysis

Samples are analysed using Orbitrap Fusion in nano-flow configuration with C18 trapping column and easy-spray PepMap column, C18, 2 μl, 100 A, 75 cm×25 cm
Resolution is performed using mobile phase A and at gradient of 5-40% of solvent B.
Data acquisition is performed
Cleaning procedure using alternate washes of 80% ACN and 10% FA is integrated into the method, and is therefore performed after each sample injection.

### Variant Identification and Targeted MS2 Analysis

Refined MVA data will be manually evaluated by examination of expression profiles of each feature. The list of features may be identified by import of Peaks Studio MS2 data if available. List of m/z values with retention time windows are exported if targeted MS2 analysis is required. The identified variants will be estimated and reported.

### Example 5: Inter-Assay Control and System Suitability Testing

In order to effectively detect low level sequence variants a control measure should be in place to ensure an adequate instrument sensitivity is achieved during each analysis. Proposed inter-assay control will consist of digested B72.3 IgG4 molecule spiked with sequence variants at level of 1% which is consistent with method's LOD.

A literature research was performed for mutations reported to occur in recombinant antibodies expressed in CHO cells. Based on the outcome, two tryptic and one chymotryptic peptide expected for B72.3 digest were selected and corresponding peptides containing amino acid mis-incorporations were synthesised by Cambridge Peptides(GPR(subS)VFPLAPCSR, VDNALQSGS(subN)SQESVTEQDSK, TADKSSR(subS)TAY). The peptides can be used to prepare the IAC samples.

The following protein mutations were reported in the literature:
Phe→Leu 11res F11L
   Zeck A, Regula J T, Larraillet V, Mautz B, Popp O, Gopfert U, et al. (2012) Low Level Sequence Variant Analysis of Recombinant Proteins: An Optimized Approach. PLoS ONE 7(7): e40328. doi:10.1371/journal.pone.0040328
   Dorai H, Sauerwald T, Campbell A, Kyung Y S, Goldstein J, et al. (2007) Investigation of Product Microheterogeneity. Bioprocess Int 5: 66-72.
Gly→Ala LC G40A
   Characterisation of TL011 Light Chain Variant, Lonza report R04760
Tyr→Gln HC Y376Q
   Harris R J, Murnane A A, Utter S L, Wagner K L, Cox E T, et al. (1993) Assessing genetic heterogeneity in production cell lines: detection by peptide mapping of a low level Tyr to Gln sequence variant in a recombinant antibody. Biotechnology 11: 1293-1297.
Ser→Arg LC S167R
Ser→Asn HC S63N
   Guo D, Gao A, Michels D A, Feeney L, Eng M, et al. (2010) Mechanisms of unintended amino acid sequence changes in recombinant monoclonal antibodies expressed in Chinese Hamster Ovary (CHO) cells. Biotechnol Bioeng 107: 163-171.
Asn→Ser multiple sites
   Khetan A, Huang Y M, Dolnikova J, Pederson N E, Wen D, et al. (2010) Control of misincorporation of serine for asparagine during antibody production using CHO cells. Biotechnol Bioeng 107: 116-123.
   Wen D, Vecchi M M, Gu S, Su L, Dolnikova J, et al. (2009) Discovery and investigation of misincorporation of serine at asparagine positions in recombinant proteins expressed in Chinese hamster ovary cells. J Biol Chem 284: 32686-32694.
Ser→Asn multiple sites
   Yu X C, Borisov O V, Alvarez M, Michels D A, Wang Y J, Ling V (2009) Identification of codon-specific serine to asparagine mistranslation in recombinant monoclonal antibodies by high-resolution mass spectrometry. Anal Chem 81: 9282-9290.

### System Suitability Test

Some variation in performance of LC-MS system was observed during method development. The differences in sensitivity and chromatographic reproducibility between assays may occur as a result of some subtle changes to the position of capillary tip, spray stability, performance of the LC system and equilibration of the easy-spray column. In order to insure an adequate system performance prior PSVA, parameters such as signal intensity, column pressure should be monitored. It is advisable to equilibrate the column by execution of around 30 blank injections to condition the column. Inter-assay control sample should be analysed before samples analysis in order to insure that suitable sensitivity is achieved.

### Example 6: Case Study Identifying Sequence Variant of Rituximab

Presented is a case study in which rituximab was used as a model protein to investigate the propensity for sequence variants to be generated in a representative Lonza cell line construction process using the GS Expression System™. Samples were analysed from cultures at early and late generation numbers, representative of typical bioproduction scenarios.

Rituximab model antibody produced at Ambr® scale from eight clonal cell lines at either early (16) or late (86) generation number were protein A purified. Duplicate lineages were generated for late generation cultures. Technical duplicates of each sample were denatured using guanidine-HCl and reduced with TCEP. Samples were digested with trypsin, lysC and chymotrypsin in separate reactions and the digests for each sample combined. LC-MS analysis performed using an Acquity UPLC and Xevo G2 QTOF mass spectrometer (Waters).

Identification of peptides displaying a difference in abundance profile across the analysis was performed by comparative analysis of the MS data with Progenesis QI software. Targeted MS/MS sequencing of the putative variant peptides was performed using an Orbitrap Fusion with identifications by use of the SPIDER algorithm within PEAKS Studio software.

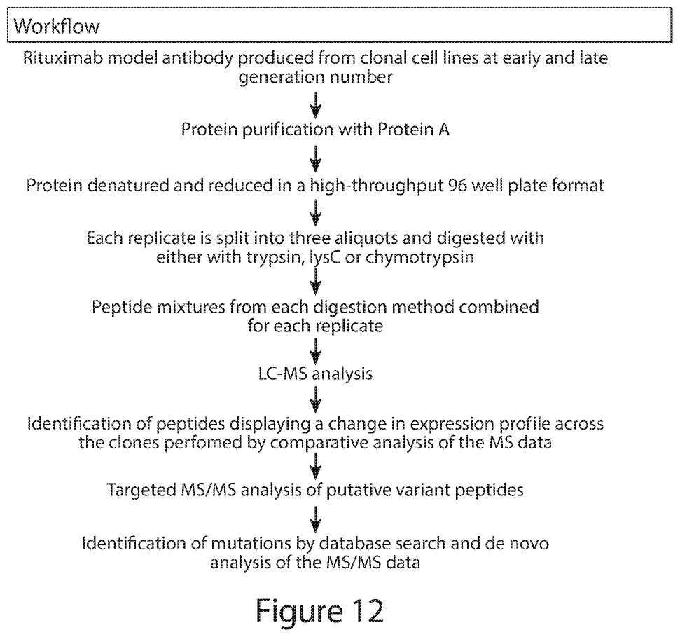

Sample processing and evaluation followed the schema of FIG. 12.

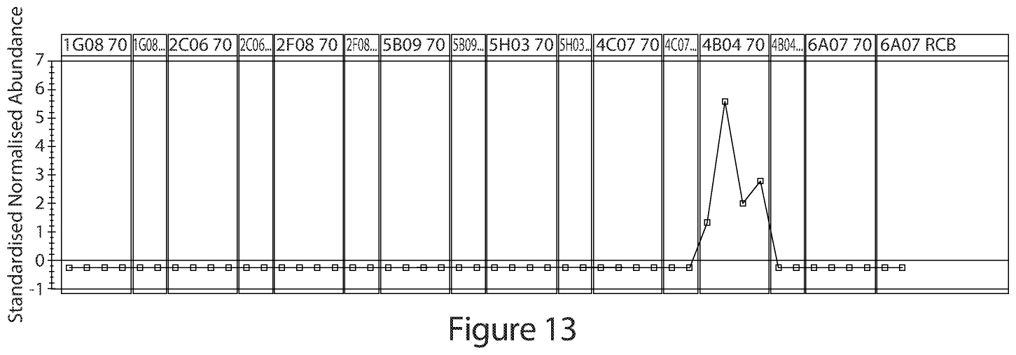

No changes in abundance profile were detected that would indicate the presence of a sequence variant in any of the early-generation research cell banks expressing the rituximab model antibody (FIG. 13). This observation suggests that the GS CHO Expression System™ is less prone to generation of these variants than some alternative expression systems, in which relatively high incidence rates have been reported. Production cell lines based on the GS CHO Expression System™ typically have low copy numbers and are selected with high stringency, making gene insertion into regions of open chromatin more probable. These factors may reduce overall risk of amino acid sequence variant incorporation via DNA mutation during cell line construction.

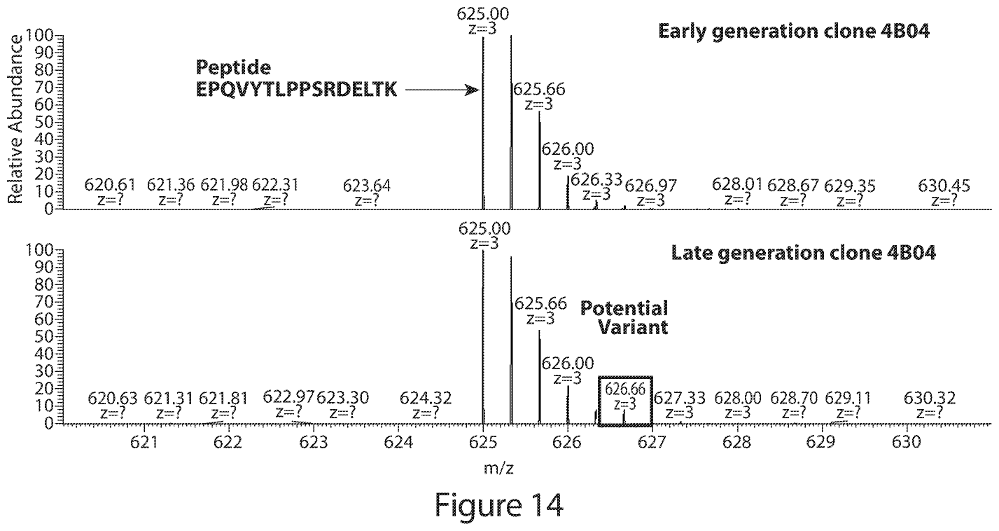

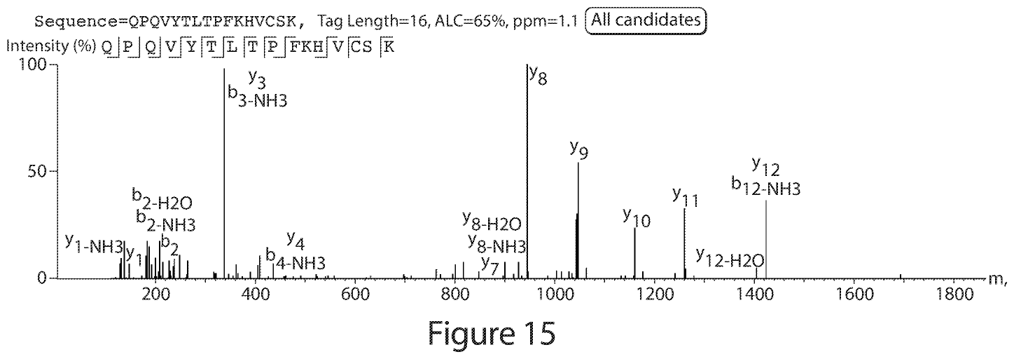

A single species was determined to be present in both late-generation lineages of the 4B04 cell line only (p<0.01) (FIG. 14). Without use of a comparative workflow, this species would be extremely difficult to detect and identify due to co-elution and isobaric mass to a ¹³C isotope of a more abundant ion. This species was not selected for MS/MS analysis using alternative DDA MS/MS-based sequence variant analysis approaches that did not include comparative assessment of MS data and was not resolved in the m/z dimension in subsequent analyses at 120,000 FWHM resolution. Targeted MS/MS analysis confirmed a proline>threonine substitution (P175T) at 1.0% and 1.7% abundance for each late-generation culture with a precision of ≤2% CV for both of these measurements (FIG. 15 and Table 6).

TABLE 6

| | Relative Abundance of Sequence Variant in Clone 4B04 | | | | | |
|---|---|---|---|---|---|---|
| | Lineage A | | | Lineage B | | |
| Generation | Protein | DNA | RNA | Protein | DNA | RNA |
| number | analysis | analysis | analysis | analysis | analysis | analysis |
| 16 | Not Detected | Not Detected | Not Detected | Not Detected | Not Detected | Not Detected |
| 86 | 1.0% | 1.1% | 2.9% | 1.7% | 2.3% | 5.7% |

Several potential mechanisms have been reported for how amino acid sequence variants can arise, including genomic DNA mutation, mistranslation at specific codons and nutrient depletion. The results were further investigated by nucleic acid analysis of early and late generation cell lines. Amplicon sequencing with molecular barcodes were performed on the genomic DNA and cDNA using Illumina MiSeq (2×300 bp). Results from DNA and RNA were combined and putative variants required data from both subsets to be rated as high confidence. Two high confidence single point mutations were identified, which were detected only in clone 4B04 late generation (both lineages). One mutation confirmed the HC P175T variant previously detected at the protein level and the other was found to be a silent mutation at R178. The mutations were found to be linked and originating from the same mutant allele. The mutant allele occurred at 1.1% in 4B04 lineage A, and at 2.3% in lineage B at DNA level. With respect to the, RNA, the frequencies were 2.9% and 5.6% respectively. These observations show that an amino acid variant species may accumulate in a production cell line as a function of cell line stability, and that this accumulation may reflect an underlying genetic instability in a clone incurred prior to the bioprocess.

The observation that an amino acid sequence variant can occur at an abundance of 1.7% in late generation cultures while remaining undetectable at early generation numbers has implications for cell line development programmes. Routine use of this analysis type in cell line stability studies is recommended to minimise overall project risk in process development.

Comparative analysis of MS data was found to be an important step for detection of a specific amino acid variant, showing that “blind spots” may be present in workflows that rely exclusively on DDA MS/MS methodology. The analytical workflow developed to support cell line development was able to effectively detect and identify low level variants, allowing removal from the clone selection process in a live cell line construction project.

### Example 7: Method Capabilities

The method was capable of detecting variants at levels of <0.1% during cell line construction testing.

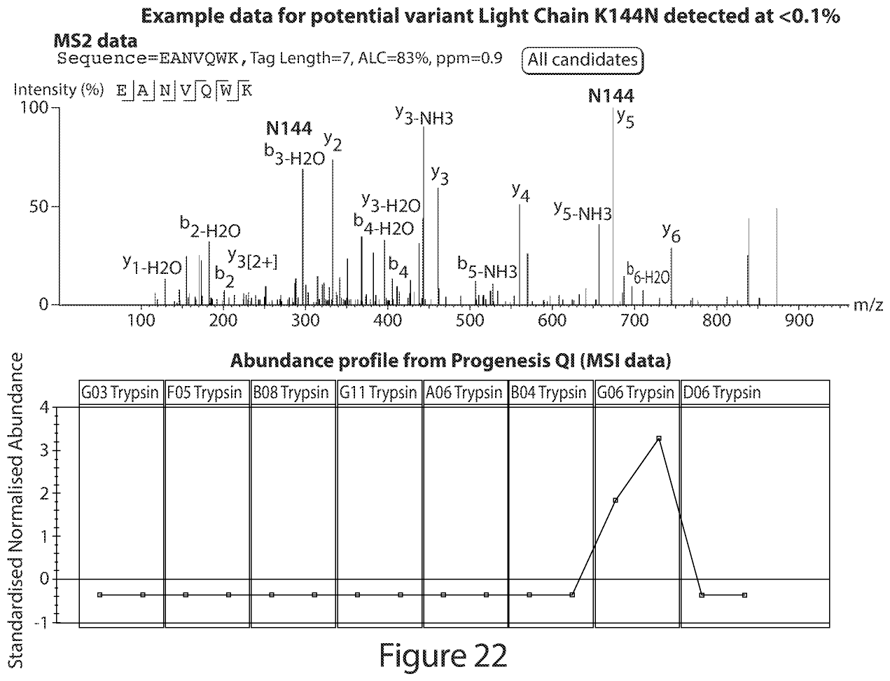

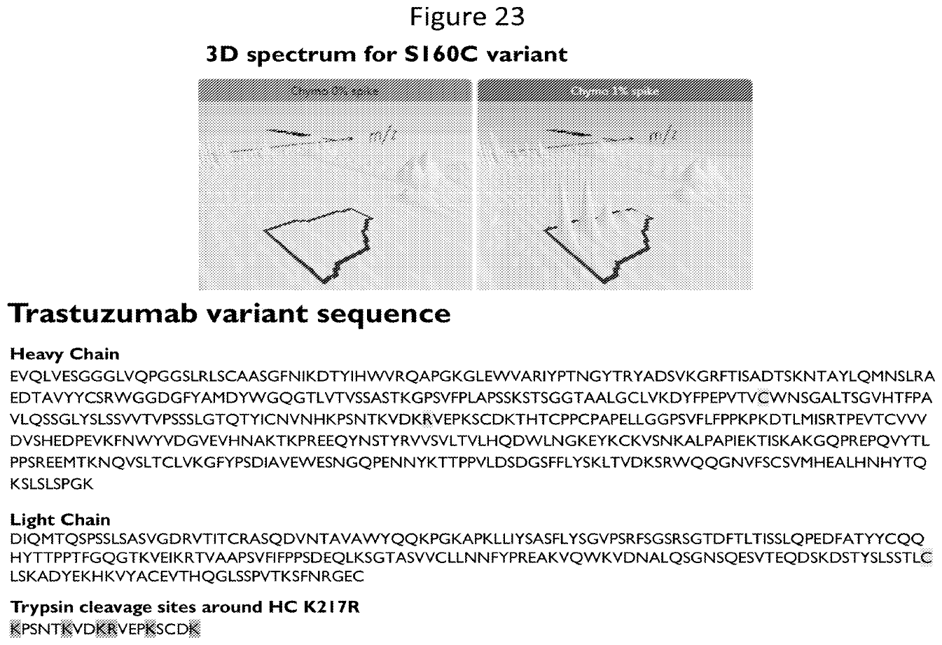

Method capability was further investigated using a spike recovery approach. Although some variants have been reported at <0.1%, the limit of detection for this method type may vary across the sequence. Samples of trastuzumab and a variant with three known residue changes (HC S160C, HC K217R and Light Chain (LC) T180C) were prepared in parallel (FIG. 22). The digested variant was spiked into the trastuzumab at 1% and 5% and analysed alongside the unspiked sample. Analysis of the spiked samples demonstrated detection in the MS1 data comparative analysis at both the 1% and 5% levels, for all three variants (FIG. 23).

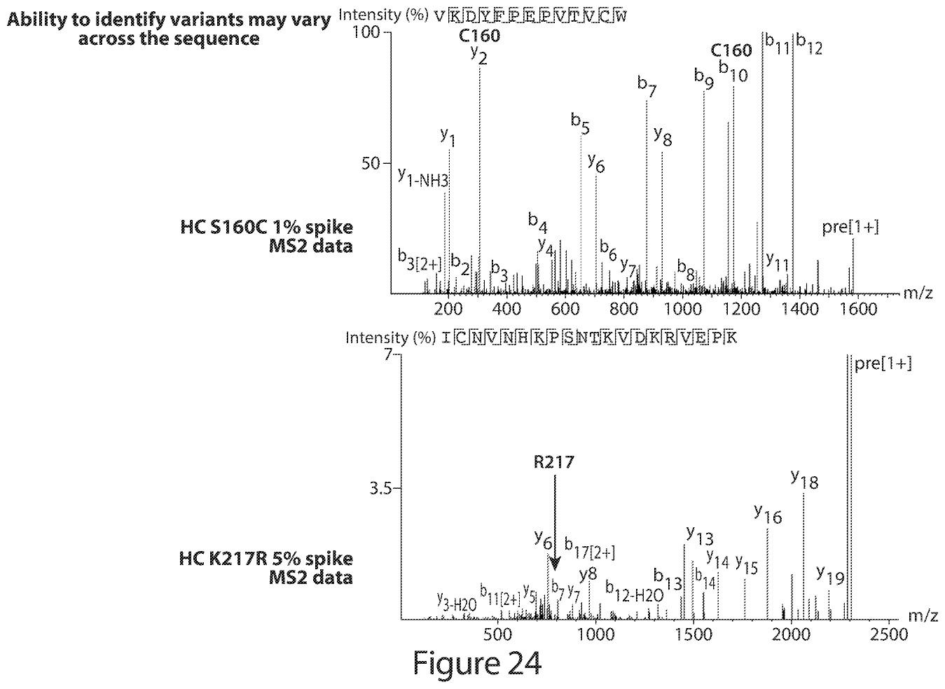

The MS2 analysis identified all of the detected variants at 5% spiking, as well as HC S160C and LC T180C at 1% (FIG. 24). The HC K217R variant was located in a lysine rich area of the sequence, with a relatively low level of redundant sequence coverage. Theoretical peptides were either extremely small or large, affecting the coverage as the small peptides were not retained on the column system. These areas may require adaptations to the analysis, such as alternative enzymes or a targeted MS2 method.

A misincorporation rate at >0.2%. for GS-CHO Xceed Expression System™ was determined at 6%. Nucleic acid analysis confirmed that a genomic mutation resulting in a variant at ≥1% at late generation may not be detectable at early generation. The limit of detection for such an analysis is not uniform across the sequence. Areas of the sequence may exhibit a higher limit of detection.

### Example 8: Case Study Testing Detection of Sequence Variants of Trastuzumab

Experiments were conducted to determine the overall rate of unintended amino acid variant incorporation within the Xceed Expression System™. Mechanisms of misincorporation and correlation with cell line stability at generation numbers representative of large-scale biomanufacturing were investigated. Finally, variability of detection limit for sequence variants at different locations within an antibody product was investigated.

Antibodies were expressed using the Xceed Expression System™ in AMBr miniature bioreactors using a platform cell culture process. Culture supernatant was purified by Protein A affinity. Samples were denatured, reduced and digested using trypsin and chymotrypsin in separate digests. The resulting peptides were separated by reverse phase chromatography at nanoflow scale and identified using an Orbitrap Fusion Q-OT-LIT mass spectrometer and a data-dependant decision tree workflow with HCD and EThcD fragmentation. Data analysis was performed using Progenesis QI for Proteomics and PEAKS Studio 7.0.

In addition to assessing data from several live development projects, method capability was determined using a spike recovery approach. Using trastuzumab as a model, three amino acid variants were expressed within a single homogenous protein, which was spiked into trastuzumab at defined relative concentrations. The ability of the method to detect each of these variants was assessed. It was determined from the development projects that many variants could be confidently detected at levels of less than 0.01%. The spike recovery approach tested the ability of the method to identify variants in possible “blind spots” where the amino acid sequence challenged this method. This was found to substantially increase the limit of detection to 1%. This observation allows additional care to be taken when assessing these regions for variants, increasing overall method robustness.

Variant incorporation as a function of cell line stability was investigated using early and late cell line generations. Three variants were detected across a number of cell line constructions that were differentially expressed at early and late generation. During further analysis of a previously reported variant, it was determined that this instability was due to a mutation in the genomic DNA, which was itself detected only in late generation cultures.

Five cell line constructions for four different products were tested to determine an interim variant rate. This resulted in a misincorporation rate of 6%, representing the percentage of cell lines that showed a variant at above 0.2% at either early or late generation for the Xceed Expression System™.

### Example 9: Case Study Investigating Misincorporation Rates

Methods

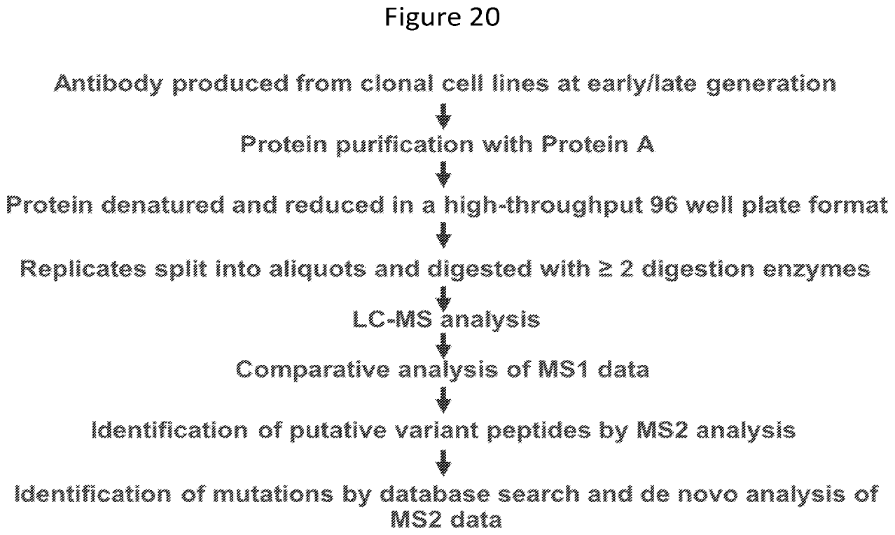

Cell culture supernatant from five cell line construction studies at Ambr® scale was Protein A purified. Studies typically consisted of eight clonal cell lines at early (˜20) and/or late (˜90) generation number (FIG. 20). Duplicates were denatured using guanidine-HCl and reduced with TCEP, then digested with a minimum of two digestion enzymes (trypsin, chymotrypsin or LysC) in separate reactions.

LC-MS analysis was performed, and MS1 data used to detect peptides displaying a difference in abundance profile across the analysis. This comparative analysis was performed using Progenesis QI software. MS2 data analysed in PEAKS Studio was used to identify the putative sequence variants.

Nucleic acid sequencing performed on genomic DNA and cDNA using Illumina MiSeq (2×300 bp). Results from DNA and RNA were combined, and putative variants required data from both subsets to be rated as high confidence.

Results

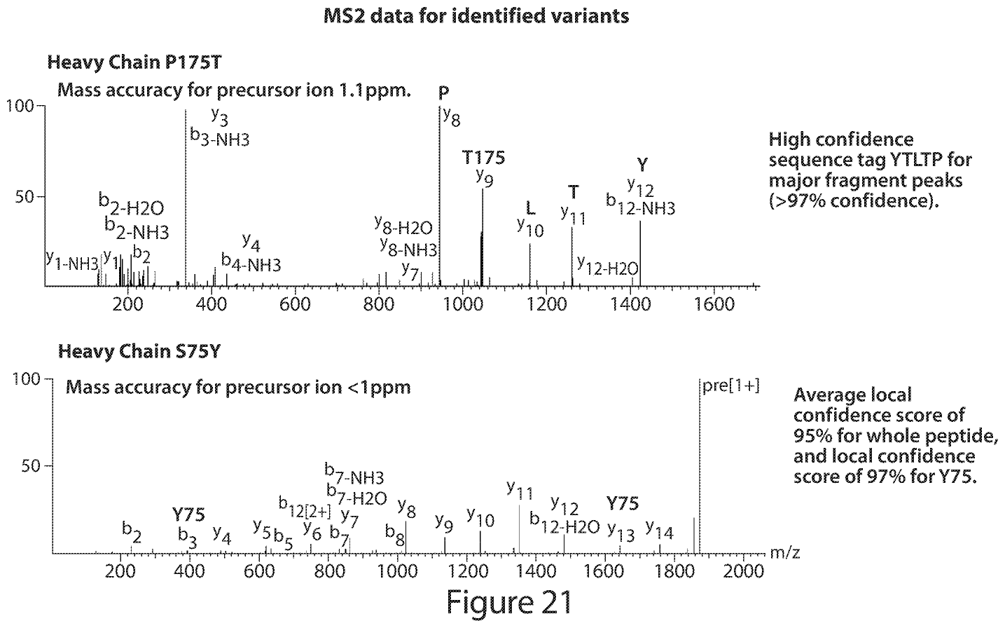

Data from analysis of five cell line constructions across four monoclonal antibody products was used to determine an interim variant rate for the GS-CHO Xceed Expression System™ (e.g., FIG. 21). A total of 34 cell lines were tested and two different variants identified at a level of >0.2% at either early or late generation. This represented an interim variant rate of 6%.

The GS CHO Expression System™ may be less prone to these variants than some alternative expression systems. Production cell lines based on this expression system typically have low copy numbers and are selected with high stringency, making gene insertion into regions of open chromatin more probable. This may reduce the risk of misincorporation via DNA mutation during cell line construction.

### Example 10: Washing Procedure

Experiments were conducted to implement a LC system wash procedure for cleaning the injection apparatus and/or trapping column in while the sample gradient is run simultaneously. The cleaning procedure must not affect the sample gradient and requires an LC system that is capable of running multiple pumps independently. Such a cleaning procedure is useful to practice in the methods of the previous Examples and in the methods taught by the present invention.

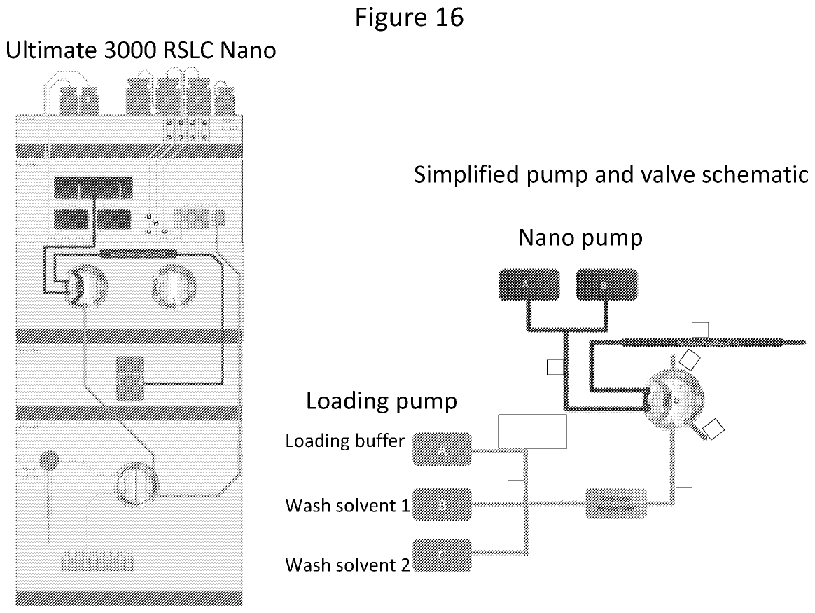

In this example, a provided LC system comprises a pump system, a switching valve to allow the pump systems to operate simultaneously, with configurations preventing cross-over of flow path, and independent programming/control of separate pumps. The present example uses an Ultimate 3000 RSLC Nano (Dionex) with separate Nanopump and Loading pump (FIG. 16), but the methods are not limited to particular equipment setups.

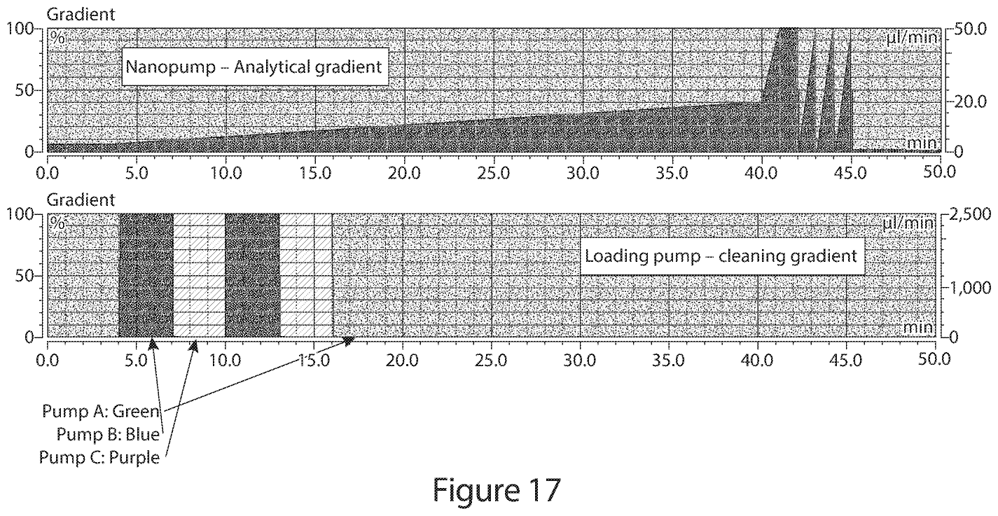

The wash sequence was determined to be alternate acidic, e.g., formic acid, and high organic, e.g., acetonitrile, washes (FIG. 17). These were utilised from lines B and C of the Loading pump. Standard solvents were used for lines A and B of the Nanopump, in accordance with the analytical separation being performed. Standard sample loading buffer was used for line A of the Loading pump, in accordance with the analytical separation being performed.

Lines A and B from the Nanopump were used to perform the analytical separation using Nanoflow. The standard analytical method was then amended to perform parallel cleaning of the injection apparatus and trap column using Lines B and C from the Loading pump.

A valve switch was added to the LC method after the sample was loaded onto the analytical column, so that the trapping column was taken out of line with the analytical column while the injection valve was in the inject position. The loading pump was used to wash the injection system and trapping column with alternate washes from loading pump lines B and C (acidic and high organic washes). The flow rate was increased from 12 μl/min to 20 μl/min for these washes. As the valve position diverted the loading pump to waste after the trapping column, the wash could be performed during the analytical gradient, which was run using the separate Nanopump. The acidic/high organic wash step was therefore run in parallel to the main method, as opposed to afterwards, so did not add to the elapsed time of the method. This parallel cleaning protocol reduces the elapsed analytical method time from about 92 minutes to about 50 minutes, a reduction of about 46%. The extra time spent cleaning (i.e. additional cleaning time), was reduced by about 84%.

A wash step for the analytical column was then added to the end of the analytical method (using the Nanopump), so that all required components had been cleaned. See Tables 7 and 8 for gradient information.

TABLE 7
Loading pump gradient

| | | | % Loading pump |
|---|---|---|---|
| RT | Flow | % Loading pump | C (high organic |
| (min) | (μl/min) | B (acidic wash) | wash) |
| 0.0 | 12 | 0 | 0 |
| 0.0 | 12 | 0 | 0 |
| 4.0 | 12 | 0 | 0 |
| 4.1 | 20 | 100 | 0 |
| 7.0 | 20 | 100 | 0 |
| 7.1 | 20 | 0 | 100 |
| 10.0 | 20 | 0 | 100 |
| 10.1 | 20 | 100 | 0 |
| 13.0 | 20 | 100 | 0 |
| 13.1 | 20 | 0 | 100 |
| 16.0 | 20 | 0 | 100 |
| 16.1 | 20 | 0 | 0 |
| 36.0 | 20 | 0 | 0 |
| 36.1 | 12 | 0 | 0 |
| 50.0 | 12 | 0 | 0 |

TABLE 8
Nanopump gradient

| | | % Nano pump B |
|---|---|---|
| RT | Flow | (analytical elution |
| (min) | (μl/min) | solvent) |
| 0.0 | 0.3 | 5 |
| 0.0 | 0.3 | 5 |
| 3.0 | 0.3 | 5 |
| 23.0 | 0.3 | 25 |
| 40.0 | 0.3 | 40 |
| 41.0 | 0.3 | 99 |
| 42.0 | 0.3 | 99 |
| 42.1 | 0.3 | 0 |
| 43.0 | 0.3 | 99 |
| 43.1 | 0.3 | 0 |
| 44.0 | 0.3 | 99 |
| 44.1 | 0.3 | 0 |
| 45.0 | 0.3 | 99 |
| 45.1 | 0.3 | 0 |
| 50.0 | 0.3 | 0 |

We claim:

1. A method of analysing a plurality of cells, the method comprising:
a) culturing a plurality of cells, at least one cell of the plurality of cells comprising a nucleic acid sequence encoding a product, said product comprising a first amino acid sequence, to make conditioned media comprising product;
b) subjecting a first sample of polypeptide from the conditioned media comprising product to a first sequence-based reaction to provide a first reaction product;
c) comparing a value for the first reaction product with a reference value, and responsive to the comparison, selecting a reaction product component for further analysis;
d) subjecting a second sample of polypeptide from the conditioned media comprising product to a second sequence-based reaction to provide a second reaction product;
e) comparing a value for the second reaction product with a reference value, and responsive to the comparison, selecting a reaction product component for further analysis;
f) optionally, subjecting a third sample of polypeptide from the conditioned media comprising product to a third sequence-based reaction to provide a third reaction product;
g) optionally, comparing a value for the third reaction product with a reference value, and responsive to the comparison, selecting a reaction product component for further analysis,
h) responsive to the results of c) and optionally e) and g), determining if a sequence other than the first amino acid sequence is present in the plurality of cells,
thereby analysing a plurality of cells.

2. The method of claim 1, comprising further culturing the plurality of cells to make second conditioned media comprising product; and performing steps b-h on the second conditioned media;
optionally further comprising culturing the plurality of cells to make third conditioned media comprising product; and performing steps b-h on the third conditioned media;
optionally further comprising culturing the plurality of cells to make a subsequent, e.g., N^{th}, wherein N=4, 5, 6, 7, 8, 9, 10, 11, 12, 13, 14, 15, 16, 17, 18, 19, or 20, conditioned media comprising product; and performing steps b-h on the subsequent, e.g., N^{th} conditioned media.

3. The method of claim 1 or 2, wherein the sample is an aliquot.

4. The method of any of claims 1-3, wherein:
(i) the plurality of conditioned culture media, the conditioned culture media, or a second, third, or subsequent, e.g., N^{th} conditioned culture media, are produced at different stages of the production of the product; or
(ii) the plurality of conditioned culture media, the conditioned culture media, or a second, third, or subsequent, e.g., N^{th} conditioned culture media are produced at different time points in the culturing of the plurality of cells.

5. The method of any of claims 1-4, comprising comparing:
i) the determination made in h) for one of a conditioned culture media, the conditioned culture media, or a second, third, or subsequent, e.g., N^{th} conditioned culture media, with
ii) the determination made in h) for another of a conditioned culture media, the conditioned culture media, or a second, third, or subsequent, e.g., N^{th} conditioned culture media.

6. The method of claim 1, further comprising analyzing a second plurality of cells, comprising performing steps a-h on the second plurality of cells;
optionally further comprising analyzing a third plurality of cells, comprising performing steps a-h on the third plurality of cells;
optionally further comprising analyzing a subsequent, e.g., N^{th}, wherein N=4, 5, 6, 7, 8, 9, 10, 11, 12, 13, 14, 15, 16, 17, 18, 19, or 20, plurality of cells, comprising performing steps a-h on the subsequent, e.g., N^{h}, plurality of cells.

7. The method of claim 6, comprising comparing:
i) the determination made in h) for one of the plurality of cells, the second, third, or subsequent, e.g., N^{th} plurality of cells, with
ii) the determination made in h) for another of the plurality of cells, second, third, or subsequent, e.g., N^{th} plurality of cells.

8. The method of claim 6 or 7, wherein each plurality of cells comprises cells of the same type, or wherein one or more, e.g., each, of the plurality of cells comprises cells of a different type.

9. The method of claim 7 or 8, comprising, responsive to the comparison, selecting a plurality of cells for producing a product comprising the first amino acid sequence.

10. The method of any of claims 1-9, wherein the first amino acid sequence corresponds to a protein product selected from Tables 1-4.

11. The method of any of claims 1-10, wherein b), d), and optionally f) comprise denaturing the sample of polypeptide.

12. The method of claim 11, wherein:
(i) denaturing the purified protein comprises incubating the purified protein in the presence of guanidine hydrochloride (GuHC1) and at an acidic pH; or
(ii) wherein denaturing the purified protein comprises incubating the purified protein in the presence of urea and deoxycholate, optionally wherein deoxycholate is precipitated out of solution prior to digestion of the purified protein product, optionally wherein (1) the deoxycholate is precipitated out of solution prior to b), prior to d), and/or optionally prior to f), or (2) the deoxycholate is precipitated out of solution prior to optionally subjecting the reaction product to a separation step.

13. The method of claim 12, wherein the deoxycholate is precipitated by the addition of an acid.

14. The method of any of claims 1-13, wherein b), d), and/or optionally f) comprise reducing the purified protein with TCEP.

15. The method of any of claims 1-13, wherein the sequence-based reaction is digestion with a proteolytic enzyme.

16. The method of claim 15, wherein the proteolytic enzyme is selected from trypsin, chymotrypsin, LysC, and AspN.

17. The method of any of claims 1-16, wherein one or more steps is performed in an apparatus suitable for high throughput sample processing; optionally wherein one or more steps is performed in a 96-well plate.

18. The method of any of claims 1-17, wherein b), d), and/or optionally f) optionally comprise a separation step comprising analyzing the reaction product using LC/MS.

19. The method of any of claims 1-18, wherein c), e), and/or optionally g) comprise identifying the amino acid sequence of a component of the reaction product identified by the comparison; optionally wherein identifying the amino acid sequence comprises using MS/MS on the component of the reaction product.

20. The method of any of claims 1-19, wherein the method is automated.

21. The method of any of claims 1-20, wherein the method employs robotic equipment.

22. The method of any of claims 1-21, wherein the method employs a micro-fluidics system.

23. The method of any of claims 1-22, further comprising, before b), d), and/or optionally f), purifying the product from the conditioned media containing product; optionally wherein purifying the product comprises using chromatography.

24. The method of any of claims 1-23, comprising a washing protocol to remove carryover contamination from equipment.

25. The method of claim 24, wherein the washing protocol comprises analyzing a blank sample using LC/MS.

26. The method of claim 24, wherein the washing protocol comprises alternate washes of acidic solution and high organic solution.

27. The method of either claim 24 or 26, wherein the washing protocol can run in parallel to the method of analyzing a plurality of cells, a method using the plurality of cells, or a polypeptide made by the plurality of cells.

28. The method of claim 27, wherein:
(i) the washing protocol does not add to the elapsed time of the method of analyzing a plurality of cells, a method using the plurality of cells, or a polypeptide made by the plurality of cells;
(ii) running the washing protocol in parallel to the method of analyzing a plurality of cells, a method using the plurality of cells, or a polypeptide made by the plurality of cells reduces the elapsed time of the method by at least about 50%, 40%, 30%, 20%, or 10%; or
(iii) running the washing protocol in parallel to the method of analyzing a plurality of cells, a method using the plurality of cells, or a polypeptide made by the plurality of cells reduces additional time spent washing by at least about 90%, 80%, 70%, 60%, 50%, 40%, 30%, 20%, or 10%.

29. The method of any of claims 1-28, further comprising evaluating the immunogenicity of the sequence other than the first amino acid sequence detected in part h).

30. The method of claim 29, wherein evaluating the immunogenicity comprises evaluating the sequence other than the first amino acid sequence detected in part h) using an in silico immunogenicity tool.

31. A method of detecting a protein sequence variant, the method comprising:
a) providing purified protein product from culture media comprising a population of cells, e.g., a plurality of cells, wherein the cells produce a protein product;
b) analyzing the purified protein product by mass spectrometry;
wherein a)-b) are repeated, in parallel or sequentially, for a plurality of samples within the same population of cells or different populations of cells; and
c) detecting protein sequence variants within the plurality of samples by comparing mass spectrometry data from the plurality of samples and a database of mass spectrometry data,
thereby detecting the protein sequence variant.

32. The method of claim 31, wherein:
(i) the populations of cells of the plurality are produced at different stages of the production of the product;
(ii) the populations of cells of the plurality are produced at different time points in the culturing of the plurality of cells;
(iii) each population of cells comprises cells of the same type; or
(iv) one or more, e.g., each, of the populations of cells comprises cells of a different type.

33. The method of claim 31 or 32, comprising, responsive to c), selecting a population of cells for producing the product.

34. The method of any of claims 31-33, wherein the protein product is a recombinant or therapeutic protein selected from Tables 1-4.

35. The method of any of claims 31-34, wherein preparing the purified protein product for analysis by mass spectrometry comprises denaturing the purified protein.

36. The method of claim 35, wherein denaturing the purified protein comprises incubating the purified protein in the presence of guanidine hydrochloride (GuHC1) and at an acidic pH.

37. The method of claim 35, wherein denaturing the purified protein comprises incubating the purified protein in the presence of urea and deoxycholate.

38. The method of claim 37, wherein the deoxycholate is precipitated out of solution prior to digestion of the purified protein product; or wherein the deoxycholate is precipitated out of solution prior to b).

39. The method of claim 37 or 38, wherein the deoxycholate is precipitated by the addition of an acid.

40. The method of any of claims 31-39, wherein preparing the purified protein product for analysis by mass spectrometry comprises reducing the purified protein with TCEP.

41. The method of any of claims 31-40, wherein preparing the purified protein product for analysis by mass spectrometry comprises digesting the purified protein with trypsin, chymotrypsin, LysC, or AspN.

42. The method of claim 41, wherein preparing the purified protein product for analysis by mass spectrometry comprises forming a plurality of aliquots of the purified protein and digesting the aliquots with a plurality of proteases, wherein each aliquot is digested by a different protease, and wherein the protease is chosen from trypsin, chymotrypsin, LysC, or AspN; optionally wherein after digestion, the plurality of aliquots are mixed together.

43. The method of any of claims 31-42, wherein preparing the purified protein product for analysis by mass spectrometry is performed in an apparatus suitable for high throughput sample processing; optionally wherein c) is performed in a 96-well plate.

44. The method of any of claims 31-43, wherein b) comprises analyzing the prepared purified protein product using LC/MS.

45. The method of any of claims 31-44, wherein c) comprises identifying peptides displaying a change in abundance by comparative analysis of the data of the plurality of populations of cells and the mass spectrometry database.

46. The method of claim 45, wherein c) further comprises analyzing peptides displaying a change in abundance by MS/MS and identifying sequence alterations by comparing the MS/MS data to MS/MS databases.

47. The method of any of claims 31-46, wherein the method is automated.

48. The method of any of claims 31-47, wherein the method employs robotic equipment.

49. The method of any of claims 31-48, wherein the method employs a micro-fluidics system.

50. The method of any of claims 31-49, wherein purifying of protein product from the population of cells to produce the purified protein product comprises purifying the protein product using chromatography.

51. The method of any of claims 31-50, wherein b) comprises a washing protocol to remove carryover contamination.

52. The method of claim 51, wherein the washing protocol comprises analyzing a blank sample using LC/MS.

53. The method of claim 51, wherein the washing protocol comprises alternate washes of acidic solution and high organic solution.

54. The method of either claim 51 or 53, wherein the washing protocol can run in parallel to the method of detecting a protein sequence variant.

55. The method of claim 54, wherein:
(i) the washing protocol does not add to the elapsed time of the method of detecting a protein sequence variant;
(ii) running the washing protocol in parallel to the method of detecting a protein sequence variant reduces the elapsed time of the method by at least about 50%, 40%, 30%, 20%, or 10%; o
(iii) running the washing protocol in parallel to the method of detecting a protein sequence variant reduces additional time spent washing by at least about 90%, 80%, 70%, 60%, 50%, 40%, 30%, 20%, or 10%.

56. The method of any of claims 31-55, further comprising evaluating the immunogenicity of a detected protein sequence variant; optionally wherein evaluating the immunogenicity of a detected protein sequence variant comprises evaluating the protein sequence variant using an in silico immunogenicity tool.

57. The method of any of claims 1-30, wherein:
(i) the method further comprises subjecting the first, second, and/or third samples of polypeptide to additional analysis to identify, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or 0-glycosylation; N-terminal cyclisation; oxidation; or pyroglutamate formation; or
(ii) if a sequence other than the first amino acid sequence is present in the plurality of cells, subjecting the sequence other than the first amino acid sequence to additional analysis to identify, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

58. The method of any of claims 31-56, wherein the method further comprises:
d) analysing the detected protein sequence variant(s) to identify, detect, evaluate, or predict one or more of the following: immunogenicity; protein aggregation; deamidation; aspartic acid isomerisation and fragmentation; C-terminal lysine processing; Fc ADCC/CDC response, half-life, and protein A purification; free cysteine thiol groups; isoelectric point; lysine glycation; N- and/or O-glycosylation; N-terminal cyclisation; oxidation; or pyroglutamate formation.

59. A method of analysing a protein sequence variant as detected in any of claims 31-56, wherein the method further comprises one or more of the following: evaluating immunogenicity, predicting protein aggregation, e.g., propensity of protein aggregation; evaluating deamidation; detecting aspartic acid isomerisation and fragmentation; detecting C-terminal lysine processing; predicting/evaluating Fc ADCC/CDC response, half-life, and protein A purification; detecting free cysteine thiol groups; evaluating isoelectric point, detecting lysine glycation; identifying N- and/or O-glycosylation; detecting N-terminal cyclisation; detecting oxidation; or detecting pyroglutamate formation.

60. A polypeptide made by the plurality of cells of the method of any of the preceding claims.


SEQUENCE LISTING (18 sequences)

SEQ ID NO: 1 - protein, 22 aa, Artificial Sequence
  source: /note=“Description of Artificial Sequence Synthetic peptide”
GFYPSDIAVEWESNGQPENNYK

SEQ ID NO: 2 - protein, 56 aa, Artificial Sequence
  source: /note=“Description of Artificial Sequence Synthetic polypeptide”
AMDYWGQGTLVTVSSASTKGPSVFPLAPSSKSTSGGTAALGCLVKDYFPEPVTVSW

SEQ ID NO: 3 - protein, 12 aa, Artificial Sequence
  source: /note=“Description of Artificial Sequence Synthetic peptide”
GPRVFPLAPCSR

SEQ ID NO: 4 - protein, 20 aa, Artificial Sequence
  source: /note=“Description of Artificial Sequence Synthetic peptide”
VDNALQSGSSQESVTEQDSK

SEQ ID NO: 5 - protein, 10 aa, Artificial Sequence
  source: /note=“Description of Artificial Sequence Synthetic peptide”
TADKSSRTAY

SEQ ID NO: 6 - protein, 79 aa, Artificial Sequence
  source: /note=“Description of Artificial Sequence Synthetic polypeptide”
QVQLQQPGAELVKPGASVKMSCKASGYTFTSYNMHWVKQTPGRGLEWIGAIYPGNGDTSYNQKFKGKATLTADKSSSTA

SEQ ID NO: 7 - protein, 16 aa, Artificial Sequence
  source: /note=“Description of Artificial Sequence Synthetic peptide”
EPQVYTLPPSRDELTK

SEQ ID NO: 8 - protein, 16 aa, Artificial Sequence
  source: /note=“Description of Artificial Sequence Synthetic peptide”
QPQVYTLTPFKHVCSK

SEQ ID NO: 9 - protein, 5 aa, Artificial Sequence
  source: /note=“Description of Artificial Sequence Synthetic peptide”
YTLTP

SEQ ID NO: 10 - protein, 7 aa, Artificial Sequence
  source: /note=“Description of Artificial Sequence Synthetic peptide”
EANVQWK

SEQ ID NO: 11 - protein, 450 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVCWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLCLSKADYEKHKVYACEVTHQGLSSPVTKSFNRGEC

SEQ ID NO: 13 - protein, 18 aa, Artificial Sequence
  source: /note=“Description of Artificial Sequence Synthetic peptide”
KPSNTKVDKRVEPKSCDK

SEQ ID NO: 14 - protein, 13 aa, Artificial Sequence
  source: /note=“Description of Artificial Sequence Synthetic peptide”
VKDYFPEPVTVCW

SEQ ID NO: 15 - protein, 20 aa, Artificial Sequence
  source: /note=“Description of Artificial Sequence Synthetic peptide”
ICNVNHKPSNTKVDKRVEPK

SEQ ID NO: 16 - protein, 12 aa, Artificial Sequence
  source: /note=“Description of Artificial Sequence Synthetic peptide”
GPSVFPLAPCSR

SEQ ID NO: 17 - protein, 20 aa, Artificial Sequence
  source: /note=“Description of Artificial Sequence Synthetic peptide”
VDNALQSGNSQESVTEQDSK

SEQ ID NO: 18 - protein, 10 aa, Artificial Sequence
  source: /note=“Description of Artificial Sequence Synthetic peptide”
TADKSSSTAY